(19) United States
(12) Patent Application Publication
(10) Pub. No.: US 2024/0218341 A1
(43) Pub. Date: Jul. 4, 2024

(54) ENZYME COMPOSITION

(71) Applicant: DSM IP Assets B.V., HEERLEN (NL)

(72) Inventors: Maaike APPELDOORN, ECHT (NL); Wouter KROES, ECHT (NL)

(21) Appl. No.: 18/553,801

(22) PCT Filed: Apr. 5, 2022
(86) PCT No.: PCT/EP2022/058938

§ 371 Date: Oct. 3, 2023

(30) Foreign Application Priority Data
Apr. 6, 2021 (EP) 21166960.1

(51) Int. Cl. C12N 9/34; C12N 9/42; C12P 19/14

(52) U.S. Cl. CPC C12N 9/2428; C12N 9/2437; C12N 9/2445; C12P 19/14; C12P 2203/00; C12Y 302/01003; C12Y 302/01021; C12Y 302/01091

(57) ABSTRACT

The application relates to an enzyme composition, a process for the preparation thereof and the use of the enzyme composition in enzymatic hydrolysis.

### FIELD

The disclosure relates to an enzyme composition, a process for the preparation thereof and the use of the enzyme composition in enzymatic hydrolysis.

### BACKGROUND

Cellulosic material is primarily composed of cellulose and may also comprise hemicellulose and lignin. It provides an attractive platform for generating alternative energy sources to fossil fuels. The material is available in large amounts and can be converted into valuable products e.g. sugars or biofuel, such as bioethanol.

Producing fermentation products from cellulosic material is known in the art and generally includes the steps of pretreatment, hydrolysis, fermentation, and optionally recovery of the fermentation products.

During the hydrolysis, which may comprise the steps of liquefaction, pre-saccharification and/or saccharification, cellulose present in the cellulosic material is partly (typically 30 to 95%, dependable on enzyme activity and hydrolysis conditions) converted into reducing sugars by cellulolytic enzymes. The hydrolysis typically takes place during a process lasting 6 to 168 hours under elevated temperatures of 45 to 50° C. and non-sterile conditions.

Commonly, the sugars are converted into valuable fermentation products such as ethanol by microorganisms like yeast. The fermentation takes place in a separate, preferably anaerobic, process step, either in the same or in a different vessel. The temperature during fermentation is adjusted to 30 to 33° C. to accommodate growth and ethanol production by microorganisms, commonly yeasts. During the fermentation process, the remaining cellulosic material is converted into reducing sugars by the enzymes already present from the hydrolysis step, while microbial biomass and ethanol are produced. The fermentation is finished once the cellulosic material is converted into fermentable sugars and all fermentable sugars are converted into ethanol, carbon dioxide and microbial biomass. This may take up to 6 days. In general, the overall process time of hydrolysis and fermentation may amount up to 13 days.

The cost of enzyme production is a major cost factor in the overall production process of fermentation products from cellulosic material. Therefore, several approaches have been taken to decrease the costs of enzymes and enzyme compositions, for example, increasing the amount of enzymes produced by a production microorganism, modulation and construction of new and improved enzymes by mutagenesis techniques and exploration of genetic diversity.

All these approaches have not increased the enzymatic activity sufficiently to overcome the high cost of enzyme production in the overall production process of fermentation products from cellulosic material. One drawback of these approaches has been the focus on a single enzyme at a time, neglecting the synergies possible with other cellulolytic enzymes.

Several attempts have been made to develop enzyme compositions to maximize the enzymatic hydrolysis of cellulosic material. For example, WO 2011/000949 describes Talaromyces mutant strains that produce specific enzyme compositions that can be used in enzymatic hydrolysis of cellulosic material.

However, these attempts have not succeeded in developing enzyme compositions with sufficiently improved performance for the hydrolysis of cellulosic biomass.

Thus, in spite of much research effort, there remains a need for improved enzyme compositions that reduce overall production costs in the process involving hydrolysis and fermentation of cellulosic material.

### SUMMARY

An object of the disclosure is to provide an improved enzyme composition, process of making the enzyme composition and use of the enzyme composition in a process for the preparation of a sugar product and/or a fermentation product from cellulosic material.

### DETAILED DESCRIPTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e. to one or at least one) of the grammatical object of the article. By way of example, “an element” may mean one element or more than one element.

The present disclosure relates to an enzyme composition comprising glucoamylase (GA) and cellobiohydrolase I (CBHI), wherein the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.98 to 0.60.

The ratio glucoamylase (R_{GA}), which is defined as the total weight of glucoamylases in the enzyme composition divided by the total weight of glucoamylases and the total weight of cellobiohydrolases I in the enzyme composition, can be calculated by the formula: R_{GA}=total GA/(total GA+total CBHI).

In an embodiment R_{GA} is from 0.02 to 0.40. In an embodiment R_{GA} is from 0.03 to 0.34. In an embodiment R_{GA} is from 0.09 to 0.29. In an embodiment R_{GA} is from 0.12 to 0.23.

In an embodiment glucoamylase is present in an amount of 0.1% to 20% (w/w) of the total amount of protein in the enzyme composition. This means that the enzyme composition comprises glucoamylase in an amount of 0.1% to 20% (w/w) of the total amount of protein in the enzyme composition. In an embodiment glucoamylase is present in an amount of 0.1% to 19% (w/w) of the total amount of protein in the enzyme composition. In an embodiment glucoamylase is present in an amount of 0.1% to 18% (w/w) of the total amount of protein in the enzyme composition. In an embodiment glucoamylase is present in an amount of 0.1% to 17% (w/w) of the total amount of protein in the enzyme composition. In an embodiment glucoamylase is present in an amount of 0.1% to 16% (w/w) of the total amount of protein in the enzyme composition. In an embodiment glucoamylase is present in an amount of 0.1% to 15% (w/w) of the total amount of protein in the enzyme composition. In an embodiment glucoamylase is present in an amount of 0.1% to 14% (w/w) of the total amount of protein in the enzyme composition. In an embodiment glucoamylase is present in an amount of 0.1% to 13% (w/w) of the total amount of protein in the enzyme composition. In an embodiment glucoamylase is present in an amount of 0.1% to 12% (w/w) of the total amount of protein in the enzyme composition. In an embodiment glucoamylase is present in an amount of 0.1% to 11% (w/w) of the total amount of protein in the enzyme composition. In an embodiment glucoamylase is present in an amount of 0.1% to 10% (w/w) of the total amount of protein in the enzyme composition.

As described herein the enzyme composition of the present disclosure comprises glucoamylase and cellobiohydrolase 1. It is to be understood that “glucoamylase” means “at least one glucoamylase” and that “cellobiohydrolase I” means “at least one cellobiohydrolase I”. The enzyme composition of the present disclosure may thus comprise more than one glucoamylase, and/or more than one cellobiohydrolase 1. In case, there are several glucoamylases and/or several cellobiohydrolases I, R_{GA} relates to the weight of all glucoamylases in the enzyme composition divided by the total weight of all glucoamylases and all cellobiohydrolases I in the enzyme composition and R_{CBH1} relates to the weight of all cellobiohydrolases I in the enzyme composition divided by the total weight of all cellobiohydrolases I and all glucoamylases in the enzyme composition.

As used herein, glucoamylases (EC 3.2.1.3) are exoglucohydrolases that catalyse hydrolysis of α-1,4 and α-1,6 glucosidic linkages to release β-D-glucose from the non-reducing ends of starch and related poly- and oligosaccharides. They are also called amyloglucosidases, glucan 1,4-alpha-glucosidase or 1,4-alpha-D-glucan glucohydrolase. They catalyzes the release of D-glucose from the non-reducing ends of starch or related oligo- and polysaccharide molecules. The majority of glucoamylases are multidomain enzymes consisting of a catalytic domain connected to a starch binding domain by an O-glycosylated linker region of varying lengths. As used herein, glucoamylases also include alpha-glycosidases (EC 3.2.1.20).

The enzyme composition as described herein may comprise a glucoamylase comprising a GH15 glucoamylase, a GH31 glucoamylase, a GH97 glucoamylase or any combination thereof. The enzyme composition as described herein preferably comprises a glucoamylase comprising a GH15 glucoamylase. The glucoamylase as used herein belongs to the structural family GH15, GH31 or GH97. Preferably, the glucoamylase as used herein belongs to the structural family GH15.

The glucoamylase as used herein may be a fungal glucoamylase. The glucoamylase as used herein may be a glucoamylase from Aspergillus, Trichoderma, Rasamsonia, Penicillium, Rhizopus, Thermomyces, to name just a few. The glucoamylase as used herein may also be an engineered glucoamylase, such as a variant enzyme comprising one or more mutations, deletions and/or insertions.

In a preferred embodiment the glucoamylase is selected from the group consisting of (a) a glucoamylase having at least 60% sequence identity to the mature polypeptide of SEQ ID NO:2, (b) a glucoamylase encoded by a polynucleotide having at least 60% sequence identity to the mature polypeptide coding sequence of SEQ ID NO:1, and (c) a fragment of the glucoamylase of (a) or (b) that has glucoamylase activity.

The mature polypeptide of SEQ ID NO:2 comprises amino acids 21 to 643 of SEQ ID NO:2. The signal peptide comprises amino acids 1 to 20 of SEQ ID NO:2. The mature polypeptide coding sequence of SEQ ID NO:1 comprises nucleotides 61 to 1932 of SEQ ID NO:1. The signal peptide comprises nucleotides 1 to 60 of SEQ ID NO:1.

In an embodiment the glucoamylase has at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide of SEQ ID NO:2. In an embodiment the glucoamylase comprises the amino acid sequence of the mature polypeptide of SEQ ID NO:2. In an embodiment the amino acid sequence of the glucoamylase consists of the mature polypeptide of SEQ ID NO:2.

In an embodiment the glucoamylase is encoded by a polynucleotide having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide coding sequence of SEQ ID NO:1. In an embodiment the glucoamylase is encoded by a polynucleotide comprising the mature polypeptide coding sequence of SEQ ID NO:1. In an embodiment the glucoamylase is encoded by a polynucleotide consisting of SEQ ID NO:1.

Glucoamylase activity can be measured as follows. The GA activity within an enzyme compositions/cocktail is determined using p-nitrophenyl-α-D-glucopyranoside as a substrate. Enzymatic hydrolysis of the substrate results in release of p-nitrophenol (pNP) which is measured at 405 nm at alkaline conditions. A substrate solution is prepared by dissolving 2 g p-nitrophenyl-α-D-glucopyranoside per litre of 200 mM sodium acetate buffer (pH 4.3) containing 2 g Triton X-100 per L of buffer. The enzyme compositions/cocktail is properly diluted with 200 mM sodium acetate buffer (pH 4.3) containing 2 g Triton X-100 per L of buffer. Subsequently, 150 μl of the substrate solution is pre-incubated at 37° C. for 5 minutes. Next, 15 μl of the diluted enzyme compositions/cocktail is added to the pre-incubated substrate solution and incubated at 37° C. for 18.3 minutes. The reaction is stopped by addition of 60 μl of 0.3 M sodium carbonate solution and after 2 minutes the absorbance is measured at 405 nm. A control sample is prepared by pre-incubation of 150 μl substrate solution plus 60 μl of 0.3 M sodium carbonate solution at 37° C. for 5 minutes. Next 15 μl of the diluted enzyme compositions/cocktail is added and incubated at 37° C. for 18.3 minutes. The absorbance is measured at 405 nm. The GA activity is calculated as follows:

(Abs 405 nm diluted cocktail blend−Abs 405 nm control sample)*DF*1000050/(εpNP*t*C*%GA)
wherein
DF=dilution factor applied to the compositions/cocktail before starting the assay
εpNP=Molar extinction coefficient of pNP at 405 nm which is 18.0 mM⁻¹·cm⁻¹
t=incubation time in seconds, in this case 1100
C=protein content in the undiluted compositions/cocktail in mg/g broth
% GA=% (w/w) of GA determined in the enzyme compositions/cocktail as described above.

The ratio cellobiohydrolase I (R_{CBH1}), which is defined as the total weight of cellobiohydrolases I in the enzyme composition divided by the total weight of cellobiohydrolases I and the total weight of glucoamylases in the enzyme composition, can be calculated by the formula: R_{CBH1}=total CBHI/(total CBHI+total GA).

In an embodiment R_{CBH1} is from 0.60 to 0.98. In an embodiment R_{CBH1} is from 0.66 to 0.97. In an embodiment R_{CBH1} is from 0.71 to 0.91. In an embodiment R_{CBH1} is from 0.77 to 0.88.

As used herein, a cellobiohydrolase (EC 3.2.1.91) is any polypeptide which is capable of catalyzing the hydrolysis of 1,4-β-D-glucosidic linkages in cellulose or cellotetraose, releasing cellobiose from the ends of the chains. This enzyme may also be referred to as cellulase 1,4-β-cellobiosidase, 1,4-β-cellobiohydrolase, 1,4-β-D-glucan cellobiohydrolase, avicelase, exo-1,4-β-D-glucanase, exocellobiohydrolase or exoglucanase.

In an embodiment the cellobiohydrolase I comprises a GH7 cellobiohydrolase 1.

In a preferred embodiment the cellobiohydrolase I is obtained from a fungus of the genus Rasamsonia, Talaromyces, Aspergillus, Trichoderma or Penicillium. In a preferred embodiment the cellobiohydrolase I is obtained from a fungus of the species Rasamsonia emersonii, Talaromyces emersonii, Talaromyces leycettanus, Aspergillus fumigatus, Trichoderma reesei or Penicillium emersonii.

In an embodiment the enzyme composition comprises a cellobiohydrolase 1 from Aspergillus, such as Aspergillus fumigatus, such as the Cel7A CBHI disclosed in SEQ ID NO:6 in WO 2011/057140 or SEQ ID NO:6 in WO 2014/130812 or a CBHI such as disclosed in WO 2013/028928 or WO 2015/081139; from Trichoderma, such as Trichoderma reesei; from Chaetomium, such as Chaetomium thermophilum; from Talaromyces, such as Talaromyces leycettanus (e.g. such as disclosed in WO 2015/187935 or WO 2016/082771), or from Penicillium, such as Penicillium emersonii (e.g. such as disclosed in WO 2011/057140). In a preferred embodiment the enzyme composition comprises a cellobiohydrolase I from Rasamsonia, such as Rasamsonia emersonii (see WO 2010/122141).

In a preferred embodiment the cellobiohydrolase I is selected from the group consisting of (a) a cellobiohydrolase I having at least 60% sequence identity to the mature polypeptide of SEQ ID NO:4, (b) a cellobiohydrolase I encoded by a polynucleotide having at least 60% sequence identity to the mature polypeptide coding sequence of SEQ ID NO:3, and (c) a fragment of the cellobiohydrolase I of (a) or (b) that has cellobiohydrolase activity.

The mature polypeptide of SEQ ID NO:4 comprises amino acids 19 to 455 of SEQ ID NO:4. The signal peptide comprises amino acids 1 to 18 of SEQ ID NO:4. The mature polypeptide coding sequence of SEQ ID NO:3 comprises nucleotides 55 to 1368 of SEQ ID NO:3. The signal peptide comprises nucleotides 1 to 54 of SEQ ID NO:3.

In an embodiment the cellobiohydrolase I is selected from the group consisting of (a) a cellobiohydrolase I having at least 60% sequence identity to the mature polypeptide of SEQ ID NO:4, (b) a cellobiohydrolase I encoded by a polynucleotide having at least 60% sequence identity to the mature polypeptide coding sequence of SEQ ID NO:5, and (c) a fragment of the cellobiohydrolase I of (a) or (b) that has cellobiohydrolase activity.

The mature polypeptide coding sequence of SEQ ID NO:5 comprises nucleotides 55 to 1368 of SEQ ID NO:5. The signal peptide comprises nucleotides 1 to 54 of SEQ ID NO:5.

In an embodiment the cellobiohydrolase I has at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide of SEQ ID NO:4. In an embodiment the cellobiohydrolase I comprises the amino acid sequence of the mature polypeptide of SEQ ID NO:4. In an embodiment the amino acid sequence of the cellobiohydrolase I consists of the mature polypeptide of SEQ ID NO:4.

In an embodiment the cellobiohydrolase I is encoded by a polynucleotide having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide coding sequence of SEQ ID NO:3. In an embodiment the cellobiohydrolase I is encoded by a polynucleotide comprising the mature polypeptide coding sequence of SEQ ID NO:3. In an embodiment the cellobiohydrolase I is encoded by a polynucleotide consisting of SEQ ID NO:3.

In an embodiment the cellobiohydrolase I is encoded by a polynucleotide having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide coding sequence of SEQ ID NO:5. In an embodiment the cellobiohydrolase I is encoded by a polynucleotide comprising the mature polypeptide coding sequence of SEQ ID NO:5. In an embodiment the cellobiohydrolase I is encoded by a polynucleotide consisting of SEQ ID NO:5.

In an embodiment the cellobiohydrolase I is a fragment of the cellobiohydrolase I of (a) or (b) (see above) that has cellobiohydrolase activity. Cellobiohydrolase I activity can be measured as follows. The CBHI activity within enzyme compositions/cocktail is determined using p-nitrophenyl-β-cellobioside as substrate. Enzymatic hydrolysis of the substrate results in release of p-nitrophenol (pNP) which is measured at 405 nm at alkaline conditions. The CBHI activity is calculated making use of the molar extinction coefficient of p-nitrophenol at 405 nm. A substrate solution is prepared of 3 mM p-nitrophenyl-β-D-cellobioside (Sigma N5759) in 100 mM sodium acetate buffer (pH 4.5) containing 10 mM gluconolactone and 25 μl Triton X-100 per L of buffer. Subsequently, 400 μl of this substrate solution is pre-incubated at 62° C. for about 10 minutes. The enzyme compositions/cocktail are properly diluted with 100 mM sodium acetate buffer (pH 4.5) containing 10 mM gluconolactone and 25 μl Triton X-100 per L of buffer. Next, 400 μl of the diluted enzyme compositions/cocktail are combined with 400 μl pre-incubated substrate solution and incubated at 62° C. for 10 minutes while continuously shaken. The reaction is stopped by addition of 800 μl of 1 M sodium carbonate solution and vigorously mixed. A control sample is prepared by pre-incubation of 400 μl substrate solution at 62° C. for about 10 minutes after which 400 μl 100 mM sodium acetate buffer (pH 4.5) containing 10 mM gluconolactone and 25 μl Triton X-100 per L of buffer and 800 μl of 1 M sodium carbonate solution is added. This is incubated at 62° C. for 10 minutes while continuously shaken. After the incubations, the absorbance of the control sample and the diluted compositions/cocktail are measured at 405 nm. CBHI activity is calculated as follows:

(Abs 405 nm diluted cocktail blend−Abs 405 nm control sample)*DF*10000/(εpNP*t*C*%GA)
wherein
DF=dilution factor applied to the enzyme compositions/cocktail before starting the assay
εpNP=Molar extinction coefficient of pNP at 405 nm which is 18.0 mM⁻¹·cm⁻¹
t=incubation time in seconds, in this case 600
C=protein content in the undiluted cocktail blend in mg/g broth
% CBHI=% (w/w) of CBHI determined in the enzyme compositions/cocktail as described above.

In an embodiment the enzyme composition comprises cellobiohydrolase I in an amount of 15% to 45% (w/w) of the total amount of protein in the enzyme composition. This means that the cellobiohydrolase I is present in an amount of 15% to 45% (w/w) of the total amount of protein in the enzyme composition. In an embodiment the cellobiohydrolase I is present in an amount of 17% to 45% (w/w) of the total amount of protein in the enzyme composition. In an embodiment the cellobiohydrolase I is present in an amount of 20% to 45% (w/w) of the total amount of protein in the enzyme composition.

An enzyme composition as described herein may further comprise a beta-glucosidase (BG).

In an embodiment an enzyme composition as described herein comprises beta-glucosidase in an amount of 1% to 20% (w/w) of the total amount of protein in the enzyme composition. This means that the beta-glucosidase is present in an amount of 1% to 20% (w/w) of the total amount of protein in the enzyme composition. In an embodiment the beta-glucosidase is present in an amount of 2% to 15% (w/w) of the total amount of protein in the enzyme composition. In an embodiment the beta-glucosidase is present in an amount of 3% to 10% (w/w) of the total amount of protein in the enzyme composition.

As used herein, a beta-glucosidase (EC 3.2.1.21) is any polypeptide which is capable of catalysing the hydrolysis of terminal, non-reducing β-D-glucose residues with release of β-D-glucose. Such a polypeptide may have a wide specificity for β-D-glucosides and may also hydrolyze one or more of the following: a β-D-galactoside, an α-L-arabinoside, a β-D-xyloside or a β-D-fucoside. This enzyme may also be referred to as amygdalase, β-D-glucoside glucohydrolase, cellobiase or gentobiase.

In an embodiment the enzyme composition comprises a beta-glucosidase from Aspergillus, such as Aspergillus oryzae, such as the one disclosed in WO 02/095014 or the fusion protein having beta-glucosidase activity disclosed in WO 2008/057637, or Aspergillus fumigatus, such as the one disclosed as SEQ ID NO:2 in WO 2005/047499 or SEQ ID NO:5 in WO 2014/130812 or an Aspergillus fumigatus beta-glucosidase variant, such as one disclosed in WO 2012/044915, such as one with the following substitutions: F100D, S283G, N456E, F512Y (using SEQ ID NO: 5 in WO 2014/130812 for numbering), or Aspergillus aculeatus, Aspergillus niger or Aspergillus kawachi. In another embodiment the beta-glucosidase is derived from Penicillium, such as Penicillium brasilianum disclosed as SEQ ID NO:2 in WO 2007/019442, or from Trichoderma, such as Trichoderma reesei, such as ones described in U.S. Pat. Nos. 6,022,725, 6,982,159, 7,045,332, 7,005,289, US 2006/0258554 US 2004/0102619. In an embodiment even a bacterial beta-glucosidase can be used. In another embodiment the beta-glucosidase is derived from Thielavia terrestris (WO 2011/035029) or Trichophaea saccata (WO 2007/019442). In a preferred embodiment the enzyme composition comprises a beta-glucosidase from Rasamsonia, such as Rasamsonia emersonii (see WO 2012/000886 or WO 2012/000890).

In a preferred embodiment the beta-glucosidase is selected from the group consisting of (a) a beta-glucosidase having at least 60% sequence identity to the mature polypeptide of SEQ ID NO:7, (b) a beta-glucosidase encoded by a polynucleotide having at least 60% sequence identity to the mature polypeptide coding sequence of SEQ ID NO:6, and (c) a fragment of the beta-glucosidase of (a) or (b) that has beta-glucosidase activity.

The mature polypeptide of SEQ ID NO:7 comprises amino acids 20 to 858 of SEQ ID NO:7. The signal peptide comprises amino acids 1 to 19 of SEQ ID NO:7. The mature polypeptide coding sequence of SEQ ID NO:6 comprises nucleotides 58 to 2577 of SEQ ID NO:6. The signal peptide comprises nucleotides 1 to 57 of SEQ ID NO:6.

In an embodiment the beta-glucosidase has at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide of SEQ ID NO:7. In an embodiment the beta-glucosidase comprises the amino acid sequence of the mature polypeptide of SEQ ID NO:7. In an embodiment the amino acid sequence of the beta-glucosidase consists of the mature polypeptide of SEQ ID NO:7.

In an embodiment the beta-glucosidase is encoded by a polynucleotide having at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the mature polypeptide coding sequence of SEQ ID NO:6. In an embodiment the beta-glucosidase is encoded by a polynucleotide comprising the mature polypeptide coding sequence of SEQ ID NO:6. In an embodiment the beta-glucosidase is encoded by a polynucleotide consisting of SEQ ID NO:6.

Beta-glucosidase activity can be measured as follows. Beta-glucosidase activity is determined at 37° C. and pH 4.40 using para-nitrophenyl-β-D-glucopyranoside (pNP-BDG) as substrate. Enzymatic hydrolysis of pNP-beta-D-glucopyranoside results in release of para-nitrophenol (pNP) and D-glucose. Quantitatively released para-nitrophenol, determined under alkaline conditions, is a measure for enzymatic activity. After 10 minutes of incubation the reaction is stopped by adding 1 M sodium carbonate and the absorbance is determined at a wavelength of 405 nm. Beta-glucosidase activity is calculated making use of the molar extinction coefficient of para-nitrophenol. A para-nitro-phenol calibration line is prepared as follows. First a 10 mM pNP stock solution in 100 mM acetate buffer pH 4.40, comprising 0.1% BSA, is made. Subsequently, dilutions of this pNP stock are made and concentrations of 0.25, 0.40, 0.67 and 1.25 mM are obtained. Next, a substrate solution is made of 5.0 mM pNP-BDG in a 100 mM acetate buffer pH 4.40. To 3 ml substrate solution, 200 μl of the pNP dilutions and 3 ml 1 M sodium carbonate is added. The absorption of the calibration mixtures is measured at 405 nm with an acetate buffer 100 mM used as a blank measurement. The pNP content is calculated using standard calculation protocols known in the art, by plotting the OD₄₀₅ versus the concentration of the pNP calibration samples with known concentration, followed by the calculation of the concentration of the unknown enzyme composition samples using the equation generated from the calibration line. Enzyme composition samples are diluted in weight corresponding to an activity between 1.7 and 3.3 units. To 3 ml substrate solution, preheated to 37° C., 200 μl of diluted sample solution is added. This is recorded as t=0. After 10.0 minutes, the reaction is stopped by adding 3 ml 1M sodium carbonate. The beta-glucosidase activity is expressed in units per gram enzyme composition sample. One unit, referred to as BG unit, is defined as the amount of enzyme that liberates one nanomol para-nitrophenol per second from para-nitrophenyl-beta-D-glucopyranoside under the defined assay conditions (pH=4.40, T=37° C.).

In an embodiment the enzyme composition of the present disclosure may further comprises an endoglucanase. Endoglucanases are enzymes which are capable of catalyzing the endohydrolysis of 1,4-β-D-glucosidic linkages in cellulose, lichenin or cereal β-D-glucans. They belong to EC 3.2.1.4 and may also be capable of hydrolyzing 1,4-linkages in β-D-glucans also containing 1,3-linkages. Endoglucanases may also be referred to as cellulases, avicelases, β-1,4-endoglucan hydrolases, β-1,4-glucanases, carboxymethyl cellulases, celludextrinases, endo-1,4-β-D-glucanases, endo-1,4-β-D-glucanohydrolases or endo-1,4-β-glucanases.

In an embodiment the endoglucanase comprises a GH5 endoglucanase and/or a GH7 endoglucanase. This means that at least one of the endoglucanases in the enzyme composition is a GH5 endoglucanase or a GH7 endoglucanase. In case there are more endoglucanases in the enzyme composition, these endoglucanases can be GH5 endoglucanases, GH7 endoglucanases or a combination of GH5 endoglucanases and GH7 endoglucanases. In a preferred embodiment the endoglucanase comprises a GH5 endoglucanase. GH classification can be found on the CAZy website.

In an embodiment the enzyme composition comprises an endoglucanase from Trichoderma, such as Trichoderma reesei; from Aspergillus, such as Aspergillus aculeatus, Aspergillus terreus or Aspergillus kawachii; from Erwinia, such as Erwinia carotovara; from Fusarium, such as Fusarium oxysporum; from Thielavia, such as Thielavia terrestris; from Humicola, such as Humicola grisea var. thermoidea or Humicola insolens; from Melanocarpus, such as Melanocarpus albomyces; from Neurospora, such as Neurospora crassa; from Myceliophthora, such as Myceliophthora thermophila; from Cladorrhinum, such as Cladorrhinum foecundissimum; and/or from Chrysosporium, such as a strain of Chrysosporium lucknowense. In an embodiment even a bacterial endoglucanase can be used including, but not limited to, Acidothermus cellulolyticus endoglucanase (see WO 91/05039; WO 93/15186; U.S. Pat. No. 5,275,944; WO 96/02551; U.S. Pat. No. 5,536,655, WO 00/70031, WO 05/093050); Thermobifida fusca endoglucanase Ill (see WO 05/093050); and Thermobifida fusca endoglucanase V (see WO 05/093050).

In an embodiment the endoglucanase is a thermostable endoglucanase. A “thermostable” endoglucanase as used herein means that the endoglucanase has a temperature optimum in the range of 45° C. to 90° C. when activity is measured between 10-30 minutes. Thermostable endoglucanases may for example be isolated from thermophilic or thermotolerant fungi or may be designed by the skilled person and artificially synthesized. In one embodiment the thermostable endoglucanase may be isolated or obtained from thermophilic or thermotolerant filamentous fungi or isolated from non-thermophilic or non-thermotolerant fungi but are found to be thermostable. In an embodiment the thermostable endoglucanase is fungal. In an embodiment the thermostable endoglucanase is obtained from a thermophilic or thermotolerant fungus. By “thermophilic fungus” is meant a fungus that grows at a temperature of 45° C. or higher. By “themotolerant” fungus is meant a fungus that grows at a temperature of 20° C. or higher, having a maximum near 55° C.

In an embodiment the thermostable endoglucanase is obtained from a fungus of the genus including, but not limited to, Humicola, Rhizomucor, Myceliophthora, Rasamsonia, Talaromyces, Penicillium, Thermomyces, Thermoascus, Aspergillus, Scytalidium, Paecilomyces, Chaetomium, Stibella, Corynascus, Malbranchea or Thielavia. Preferred species of these genera include, but are not limited to, Humicola grisea var. thermoidea, Humicola lanuginosa, Humicola hyalothermophilia, Myceliophthora thermophila, Myceliophthora hinnulea, Rasamsonia byssochlamydoides, Rasamsonia emersonii, Rasamsonia argillacea, Rasamsonia eburnean, Rasamsonia brevistipitata, Rasamsonia cylindrospora, Rhizomucor pusillus, Rhizomucor miehei, Talaromyces bacillisporus, Talaromyces leycettanus, Talaromyces thermophilus, Talaromyces emersonii, Thermomyces lenuginosus, Thermomyces stellatus, Thermoascus crustaceus, Thermoascus thermophilus, Thermoascus aurantiacus, Penicillium emersonii, Penicillium cylindrosporum, Aspergillus terreus, Aspergillus fumigatus, Scytalidium thermophilum, Paecilomyces byssochlamydoides, Chaetomium thermophilum, Chaetomium olivicolor, Stibella thermophila, Corynascus sepedonium, Malbranchea cinnamonmea and Thielavia terrestris.

In a preferred embodiment the thermostable endoglucanase is obtained from a fungus of the genus Rasamsonia, Talaromyces, Thermoascus or Penicillium.

In an embodiment the endoglucanase is a fragment of an endoglucanase as described herein that has endoglucanase activity. Endoglucanase activity can be measured as follows. Endoglucanase activity is determined at 62° C. and pH 4.5 using AZO-carboxymethyl cellulose (AZO-CMC) as substrate. Enzymatic hydrolysis of AZO-CMC results in release of low-molecular weight dyed fragments, which remain in solution on addition of a precipitant solution to the reaction mixture. Centrifugation removes the insoluble high-molecular weight material and the colour of the supernatant is a measure of EG-activity. The substrate solution is made by dissolving 2 g AZO-CMC powder in 80 ml hot milliQ-water (±95° C.) which is stirred for about 20 minutes to become homogenous. Subsequently, 5 ml acetate buffer (2 M, pH 4.5) is added and the substrate solution is made up till 100 ml with milliQ-water. The precipitation solution is made by dissolving 40 g sodium acetate and 4 g of zinc acetate dihydrate in 150 mL of milliQ-water. The pH is adjusted to 5.0 with 4 M HCl and the final solution is made up to 200 ml with milliQ-water. Before use, 20 mL of this solution is mixed with 80 mL ethanol (96%) resulting in the final precipitation solution. Enzyme samples are diluted based on weight in sodium acetate buffer (100 mM, pH 4.5, containing ±25 μL Triton X-100/L) corresponding to a final absorption between 0.15 and 1.0 AU in the assay. The substrate solution (200 μL) is pre-heated in a 2 ml eppendorf tube using a thermomixer at 62° C. and 800 rpm for 10 minutes. Subsequently, 200 μl diluted enzyme sample is added and the reaction mixture is incubated another 10 minutes at 62° C. The reaction is stopped by adding 1 mL of precipitation solution. The reaction mixture is mixed and equilibrated at room temperature for 10 minutes. After this, the reaction mixture is mixed again and centrifuged at 1000×g for 10 minutes at room temperature. The absorbance of the supernatant is measured at 590 nm using water to calibrate the spectrophotometer to zero. A blank is prepared in the same way as described for the enzyme samples above, only instead of adding diluted enzyme sample, sodium acetate buffer (100 mM, pH 4.5, containing ±25 μL Triton X-100/L)) is added. The endoglucanase activity is expressed in units per mg protein. One EG-unit is defined as the amount of enzyme that results in 1 mAU increase per second from AZO-CMC measured at 590 nm at the assay conditions described (pH 4.5, 62° C., 10 minutes incubation).

In an embodiment the enzyme composition of the present disclosure may further comprises a hemicellulase. As described herein the enzyme composition of the present disclosure preferably comprises a hemicellulase. It is to be understood that “a hemicellulase” means “at least one hemicellulase”. The enzyme composition of the present disclosure may thus comprise more than one hemicellulase. In an embodiment the hemicellulase comprises a beta-xylosidase and/or an endoxylanase.

As used herein, beta-xylosidases (EC 3.2.1.37) are polypeptides which are capable of catalysing the hydrolysis of 1,4-β-D-xylans, to remove successive D-xylose residues from the non-reducing termini. Beta-xylosidases may also hydrolyze xylobiose. Beta-xylosidase may also be referred to as xylan 1,4-β-xylosidase, 1,4-β-D-xylan xylohydrolase, exo-1,4-β-xylosidase or xylobiase.

In an embodiment the beta-xylosidase comprises a GH3 beta-xylosidase. This means that at least one of the beta-xylosidases in the enzyme composition is a GH3 beta-xylosidase. In an embodiment all beta-xylosidases in the enzyme composition are GH3 beta-xylosidases.

In an embodiment the enzyme composition comprises a beta-xylosidase from Neurospora crassa, Aspergillus fumigatus or Trichoderma reesei. In a preferred embodiment the enzyme composition comprises a beta-xylosidase from Rasamsonia, such as Rasamsonia emersonii(see WO 2014/118360).

As used herein, an endoxylanase (EC 3.2.1.8) is any polypeptide which is capable of catalysing the endohydrolysis of 1,4-β-D-xylosidic linkages in xylans. This enzyme may also be referred to as endo-1,4-β-xylanase or 1,4-β-D-xylan xylanohydrolase. An alternative is EC 3.2.1.136, a glucuronoarabinoxylan endoxylanase, an enzyme that is able to hydrolyze 1,4 xylosidic linkages in glucuronoarabinoxylans.

In an embodiment the endoxylanase comprises a GH10 xylanase. This means that at least one of the endoxylanases in the enzyme composition is a GH10 xylanase. In an embodiment all endoxylanases in the enzyme composition are GH10 xylanases.

In an embodiment the enzyme composition comprises an endoxylanase from Aspergillus aculeatus (see WO 94/21785), Aspergillus fumigatus (see WO 2006/078256), Penicillium pinophilum (see WO 2011/041405), Penicillium sp. (see WO 2010/126772), Thielavia terrestris NRRL 8126 (see WO 2009/079210), Talaromyces leycettanus, Thermobifida fusca, or Trichophaea saccata GH10 (see WO 2011/057083). In a preferred embodiment the enzyme composition comprises an endoxylanase from Rasamsonia, such as Rasamsonia emersonii(see WO 02/24926).

In an embodiment an enzyme composition as described herein further comprises a lytic polysaccharide monooxygenase (LPMO) and/or a cellobiohydrolase II (CBHII).

As used herein, lytic polysaccharide monooxygenases are enzymes that have recently been classified by CAZy in family AA9 (Auxiliary Activity Family 9) or family AA10 (Auxiliary Activity Family 10). Ergo, there exist AA9 lytic polysaccharide monooxygenases and AA10 lytic polysaccharide monooxygenases. Lytic polysaccharide monooxygenases are able to open a crystalline glucan structure and enhance the action of cellulases on lignocellulose substrates. They are enzymes having cellulolytic enhancing activity. Lytic polysaccharide monooxygenases may also affect cello-oligosaccharides. According to the latest literature, (see Isaksen et al., Journal of Biological Chemistry, vol. 289, no. 5, p. 2632-2642), proteins named GH61 (glycoside hydrolase family 61 or sometimes referred to EGIV) are lytic polysaccharide monooxygenases. GH61 was originally classified as endoglucanase based on measurement of very weak endo-1,4-β-d-glucanase activity in one family member but have recently been reclassified by CAZy in family AA9. CBM33 (family 33 carbohydrate-binding module) is also a lytic polysaccharide monooxygenase (see Isaksen et al, Journal of Biological Chemistry, vol. 289, no. 5, pp. 2632-2642). CAZy has recently reclassified CBM33 in the AA10 family.

In an embodiment the lytic polysaccharide monooxygenase comprises an AA9 lytic polysaccharide monooxygenase. This means that at least one of the lytic polysaccharide monooxygenases in the enzyme composition is an AA9 lytic polysaccharide monooxygenase. In an embodiment, all lytic polysaccharide monooxygenases in the enzyme composition are AA9 lytic polysaccharide monooxygenase.

In an embodiment the enzyme composition comprises a lytic polysaccharide monooxygenase from Thermoascus, such as Thermoascus aurantiacus, such as the one described in WO 2005/074656 as SEQ ID NO:2 and SEQ ID NO:1 in WO2014/130812 and in WO 2010/065830; or from Thielavia, such as Thielavia terrestris, such as the one described in WO 2005/074647 as SEQ ID NO: 8 or SEQ ID NO:4 in WO2014/130812 and in WO 2008/148131, and WO 2011/035027; or from Aspergillus, such as Aspergillus fumigatus, such as the one described in WO 2010/138754 as SEQ ID NO:2 or SEQ ID NO: 3 in WO2014/130812; or from Penicillium, such as Penicillium emersonii, such as the one disclosed as SEQ ID NO:2 in WO 2011/041397 or SEQ ID NO:2 in WO2014/130812. Other suitable lytic polysaccharide monooxygenases include, but are not limited to, Trichoderma reesei (see WO 2007/089290), Myceliophthora thermophila (see WO 2009/085935, WO 2009/085859, WO 2009/085864, WO 2009/085868), Penicillium pinophilum (see WO 2011/005867), Thermoascus sp. (see WO 2011/039319), and Thermoascus crustaceous (see WO 2011/041504). Other cellulolytic enzymes that may be comprised in the enzyme composition are described in WO 98/13465, WO 98/015619, WO 98/015633, WO 99/06574, WO 99/10481, WO 99/025847, WO 99/031255, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. Nos. 5,457,046, 5,648,263, and 5,686,593, to name just a few. In a preferred embodiment, the lytic polysaccharide monooxygenase is from Rasamsonia, e.g. Rasamsonia emersonii (see WO 2012/000892).

In an embodiment the enzyme composition comprises a cellobiohydrolase II from Aspergillus, such as Aspergillus fumigatus, such as the one in SEQ ID NO:7 in WO 2014/130812 or from Trichoderma, such as Trichoderma reesei, or from Talaromyces, such as Talaromyces leycettanus, or from Thielavia, such as Thielavia terrestris, such as cellobiohydrolase II CEL6A from Thielavia terrestris. In a preferred embodiment the enzyme composition comprises a cellobiohydrolase II from Rasamsonia, such as Rasamsonia emersonii (see WO 2011/098580).

An enzyme composition comprises preferably at least two activities, although typically a composition will comprise more than two activities, for example, three, four, five, six, seven, eight, nine or even more activities. An enzyme composition may comprise more than one enzyme activity per activity class. An enzyme composition may comprise one type of cellulase activity and/or hemicellulase activity and/or pectinase activity.

In an embodiment the enzyme composition comprises at least two cellulases. As used herein, a cellulase is any polypeptide which is capable of degrading or modifying cellulose. The at least two cellulases may contain the same or different activities. The enzyme composition may also comprise at least one enzyme other than a cellulase, e.g. a hemicellulase or a pectinase. As used herein, a hemicellulase is any polypeptide which is capable of degrading or modifying hemicellulose. As used herein, a pectinase is any polypeptide which is capable of degrading or modifying pectin. The at least one other enzyme may have an auxiliary enzyme activity, i.e. an additional activity which, either directly or indirectly leads to lignocellulose degradation. Examples of such auxiliary activities are mentioned herein.

In an embodiment the enzyme composition as described herein comprises in addition to glucoamylase (GA) and cellobiohydrolase I (CBHI) one, two, three, four classes or more of cellulase, for example one, two, three or four or all of a lytic polysaccharide monooxygenase (LPMO), an endoglucanase (EG), a cellobiohydrolase II (CBHII) and a beta-glucosidase (BG).

In an embodiment the enzyme composition as described herein comprises a glucoamylase (GA), a cellobiohydrolase I, a lytic polysaccharide monooxygenase, an endoglucanase, a cellobiohydrolase II, a beta-glucosidase, a beta-xylosidase and an endoxylanase.

In an embodiment the enzyme composition also comprises one or more of the below mentioned enzymes.

As used herein, a β-(1,3)(1,4)-glucanase (EC 3.2.1.73) is any polypeptide which is capable of catalysing the hydrolysis of 1,4-β-D-glucosidic linkages in β-D-glucans containing 1,3- and 1,4-bonds. Such a polypeptide may act on lichenin and cereal β-D-glucans, but not on β-D-glucans containing only 1,3- or 1,4-bonds. This enzyme may also be referred to as licheninase, 1,3-1,4-β-D-glucan 4-glucanohydrolase, β-glucanase, endo-β-1,3-1,4 glucanase, lichenase or mixed linkage β-glucanase. An alternative for this type of enzyme is EC 3.2.1.6, which is described as endo-1,3(4)-beta-glucanase. This type of enzyme hydrolyses 1,3- or 1,4-linkages in beta-D-glucanse when the glucose residue whose reducing group is involved in the linkage to be hydrolysed is itself substituted at C-3. Alternative names include endo-1,3-beta-glucanase, laminarinase, 1,3-(1,3;1,4)-beta-D-glucan 3 (4) glucanohydrolase. Substrates include laminarin, lichenin and cereal beta-D-glucans.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase. Examples of arabinofuranosidases that may be comprised in the enzyme composition include, but are not limited to, arabinofuranosidases from Aspergillus niger, Humicola insolens DSM 1800 (see WO 2006/114094 and WO 2009/073383) and M. giganteus (see WO 2006/114094).

As used herein, an α-D-glucuronidase (EC 3.2.1.139) is any polypeptide which is capable of catalysing a reaction of the following form: alpha-D-glucuronoside+H(2)O=an alcohol+D-glucuronate. This enzyme may also be referred to as alpha-glucuronidase or alpha-glucosiduronase. These enzymes may also hydrolyse 4-O-methylated glucoronic acid, which can also be present as a substituent in xylans. An alternative is EC 3.2.1.131: xylan alpha-1,2-glucuronosidase, which catalyses the hydrolysis of alpha-1,2-(4-O-methyl)glucuronosyl links. Examples of alpha-glucuronidases that may be comprised in the enzyme composition include, but are not limited to, alpha-glucuronidases from Aspergillus clavatus, Aspergillus fumigatus, Aspergillus niger, Aspergillus terreus, Humicola insolens (see WO 2010/014706), Penicillium aurantiogriseum (see WO 2009/068565) and Trichoderma reesei.

As used herein, an acetyl xylan esterase (EC 3.1.1.72) is any polypeptide which is capable of catalysing the deacetylation of xylans and xylo-oligosaccharides. Such a polypeptide may catalyze the hydrolysis of acetyl groups from polymeric xylan, acetylated xylose, acetylated glucose, alpha-napthyl acetate or p-nitrophenyl acetate but, typically, not from triacetylglycerol. Such a polypeptide typically does not act on acetylated mannan or pectin. Examples of acetylxylan esterases that may be comprised in the enzyme composition include, but are not limited to, acetylxylan esterases from Aspergillus aculeatus (see WO 2010/108918), Chaetomium globosum, Chaetomium gracile, Humicola insolens DSM 1800 (see WO 2009/073709), Hypocrea jecorina (see WO 2005/001036), Myceliophtera thermophila (see WO 2010/014880), Neurospora crassa, Phaeosphaeria nodorum and Thielavia terrestris NRRL 8126 (see WO 2009/042846). In a preferred embodiment the enzyme composition comprises an acetyl xylan esterase from Rasamsonia, such as Rasamsonia emersonii (see WO 2010/000888)

As used herein, a feruloyl esterase (EC 3.1.1.73) is any polypeptide which is capable of catalysing a reaction of the form: feruloyl-saccharide+H₂O=ferulate+saccharide. The saccharide may be, for example, an oligosaccharide or a polysaccharide. It may typically catalyse the hydrolysis of the 4-hydroxy-3-methoxycinnamoyl (feruloyl) group from an esterified sugar, which is usually arabinose in ‘natural’ substrates. p-nitrophenol acetate and methyl ferulate are typically poorer substrates. This enzyme may also be referred to as cinnamoyl ester hydrolase, ferulic acid esterase or hydroxycinnamoyl esterase. It may also be referred to as a hemicellulase accessory enzyme, since it may help xylanases and pectinases to break down plant cell wall hemicellulose and pectin. Examples of feruloyl esterases (ferulic acid esterases) that may be comprised in the enzyme composition include, but are not limited to, feruloyl esterases form Humicola insolens DSM 1800 (see WO 2009/076122), Neosartorya fischeri, Neurospora crassa, Penicillium aurantiogriseum (see WO 2009/127729), and Thielavia terrestris (see WO 2010/053838 and WO 2010/065448).

As used herein, a coumaroyl esterase (EC 3.1.1.73) is any polypeptide which is capable of catalysing a reaction of the form: coumaroyl-saccharide+H(2)O=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. This enzyme also falls within EC 3.1.1.73 so may also be referred to as a feruloyl esterase.

As used herein, an α-galactosidase (EC 3.2.1.22) is any polypeptide which is capable of catalysing the hydrolysis of terminal, non-reducing α-D-galactose residues in α-D-galactosides, including galactose oligosaccharides, galactomannans, galactans and arabinogalactans. Such a polypeptide may also be capable of hydrolyzing α-D-fucosides. This enzyme may also be referred to as melibiase.

As used herein, a β-galactosidase (EC 3.2.1.23) is any polypeptide which is capable of catalysing the hydrolysis of terminal non-reducing β-D-galactose residues in β-D-galactosides. Such a polypeptide may also be capable of hydrolyzing α-L-arabinosides. This enzyme may also be referred to as exo-(1->4)-β-D-galactanase or lactase.

As used herein, a β-mannanase (EC 3.2.1.78) is any polypeptide which is capable of catalysing the random hydrolysis of 1,4-β-D-mannosidic linkages in mannans, galactomannans and glucomannans. This enzyme may also be referred to as mannan endo-1,4-β-mannosidase or endo-1,4-mannanase.

As used herein, a β-mannosidase (EC 3.2.1.25) is any polypeptide which is capable of catalysing the hydrolysis of terminal, non-reducing β-D-mannose residues in β-D-mannosides. This enzyme may also be referred to as mannanase or mannase.

As used herein, an endo-polygalacturonase (EC 3.2.1.15) is any polypeptide which is capable of catalysing the random hydrolysis of 1,4-α-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-α-1,4-galacturonide glycanohydrolase, endogalacturonase; endo-D-galacturonase or poly(1,4-α-D-galacturonide) glycanohydrolase.

As used herein, a pectin methyl esterase (EC 3.1.1.11) is any enzyme which is capable of catalysing the reaction: pectin+n H₂O=n methanol+pectate. The enzyme may also be known as pectinesterase, pectin demethoxylase, pectin methoxylase, pectin methylesterase, pectase, pectinoesterase or pectin pectylhydrolase.

As used herein, an endo-galactanase (EC 3.2.1.89) is any enzyme capable of catalysing the endohydrolysis of 1,4-β-D-galactosidic linkages in arabinogalactans. The enzyme may also be known as arabinogalactan endo-1,4-β-galactosidase, endo-1,4-β-galactanase, galactanase, arabinogalactanase or arabinogalactan 4-β-D-galactanohydrolase.

As used herein, a pectin acetyl esterase is defined herein as any enzyme which has an acetyl esterase activity which catalyses the deacetylation of the acetyl groups at the hydroxyl groups of GaIUA residues of pectin.

As used herein, an endo-pectin lyase (EC 4.2.2.10) is any enzyme capable of catalysing the eliminative cleavage of (1→4)-α-D-galacturonan methyl ester to give oligosaccharides with 4-deoxy-6-O-methyl-α-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-α-D-galacturonan lyase.

As used herein, a pectate lyase (EC 4.2.2.2) is any enzyme capable of catalysing the eliminative cleavage of (1→4)-α-D-galacturonan to give oligosaccharides with 4-deoxy-α-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, α-1,4-D-endopolygalacturonic acid lyase, PGA lyase, PPase-N, endo-α-1,4-polygalacturonic acid lyase, polygalacturonic acid lyase, pectin trans-eliminase, polygalacturonic acid trans-eliminase or (1→4)-α-D-galacturonan lyase.

As used herein, an alpha rhamnosidase (EC 3.2.1.40) is any polypeptide which is capable of catalysing the hydrolysis of terminal non-reducing α-L-rhamnose residues in α-L-rhamnosides or alternatively in rhamnogalacturonan. This enzyme may also be known as α-L-rhamnosidase T, α-L-rhamnosidase N or α-L-rhamnoside rhamnohydrolase.

As used herein, exo-galacturonase (EC 3.2.1.82) is any polypeptide capable of hydrolysis of pectic acid from the non-reducing end, releasing digalacturonate. The enzyme may also be known as exo-poly-α-galacturonosidase, exopolygalacturonosidase or exopolygalacturanosidase.

As used herein, exo-galacturonase (EC 3.2.1.67) is any polypeptide capable of catalysing: (1,4-α-D-galacturonide)ₙ+H₂O=(1,4-α-D-galacturonide)ₙ₋₁+D-galacturonate. The enzyme may also be known as galacturan 1,4-α-galacturonidase, exopolygalacturonase, poly(galacturonate) hydrolase, exo-D-galacturonase, exo-D-galacturonanase, exopoly-D-galacturonase or poly(1,4-α-D-galacturonide) galacturonohydrolase.

As used herein, exopolygalacturonate lyase (EC 4.2.2.9) is any polypeptide capable of catalysing eliminative cleavage of 4-(4-deoxy-α-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-α-D-galacturonan reducing-end-disaccharide-lyase.

As used herein, rhamnogalacturonan hydrolase is any polypeptide which is capable of hydrolyzing the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

As used herein, rhamnogalacturonan lyase is any polypeptide which is any polypeptide which is capable of cleaving α-L-Rhap-(1→4)-α-D-GalpA linkages in an endo-fashion in rhamnogalacturonan by beta-elimination.

As used herein, rhamnogalacturonan acetyl esterase is any polypeptide which catalyzes the deacetylation of the backbone of alternating rhamnose and galacturonic acid residues in rhamnogalacturonan.

As used herein, rhamnogalacturonan galacturonohydrolase is any polypeptide which is capable of hydrolyzing galacturonic acid from the non-reducing end of strictly alternating rhamnogalacturonan structures in an exo-fashion.

As used herein, xylogalacturonase is any polypeptide which acts on xylogalacturonan by cleaving the β-xylose substituted galacturonic acid backbone in an endo-manner. This enzyme may also be known as xylogalacturonan hydrolase.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase.

As used herein, endo-arabinanase (EC 3.2.1.99) is any polypeptide which is capable of catalysing endohydrolysis of 1,5-α-arabinofuranosidic linkages in 1,5-arabinans. The enzyme may also be known as endo-arabinase, arabinan endo-1,5-α-L-arabinosidase, endo-1,5-α-L-arabinanase, endo-α-1,5-arabanase; endo-arabanase or 1,5-α-L-arabinan 1,5-α-L-arabinanohydrolase.

“Protease” includes enzymes that hydrolyze peptide bonds (peptidases), as well as enzymes that hydrolyze bonds between peptides and other moieties, such as sugars (glycopeptidases). Many proteases are characterized under EC 3.4 and are suitable for use in the processes as described herein. Some specific types of proteases include, cysteine proteases including pepsin, papain and serine proteases including chymotrypsins, carboxypeptidases and metalloendopeptidases.

“Lipase” includes enzymes that hydrolyze lipids, fatty acids, and acylglycerides, including phospoglycerides, lipoproteins, diacylglycerols, and the like. In plants, lipids are used as structural components to limit water loss and pathogen infection. These lipids include waxes derived from fatty acids, as well as cutin and suberin.

“Ligninase” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin. Ligninases include but are not limited to the following group of enzymes: lignin peroxidases (EC 1.11.1.14), manganese peroxidases (EC 1.11.1.13), laccases (EC 1.10.3.2) and feruloyl esterases (EC 3.1.1.73).

“Hexosyltransferase” (2.4.1-) includes enzymes which are capable of catalysing a transferase reaction, but which can also catalyze a hydrolysis reaction, for example of cellulose and/or cellulose degradation products. An example of a hexosyltransferase which may be used is a β-glucanosyltransferase. Such an enzyme may be able to catalyze degradation of (1,3)(1,4)glucan and/or cellulose and/or a cellulose degradation product.

“Glucuronidase” includes enzymes that catalyze the hydrolysis of a glucuronoside, for example β-glucuronoside to yield an alcohol. Many glucuronidases have been characterized and may be suitable for use, for example β-glucuronidase (EC 3.2.1.31), hyalurono-glucuronidase (EC 3.2.1.36), glucuronosyl-disulfoglucosamine glucuronidase (3.2.1.56), glycyrrhizinate β-glucuronidase (3.2.1.128) or α-D-glucuronidase (EC 3.2.1.139).

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. As described herein, an expansin-like protein or swollenin-like protein may comprise one or both of such domains and/or may disrupt the structure of cell walls (such as disrupting cellulose structure), optionally without producing detectable amounts of reducing sugars.

A cellulose induced protein, for example the polypeptide product of the cip1 or cip2 gene or similar genes (see Foreman et al., J. Biol. Chem. 278(34), 31988-31997, 2003), a cellulose/cellulosome integrating protein, for example the polypeptide product of the cipA or cipC gene, or a scaffoldin or a scaffoldin-like protein. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module (CBM) that mediates attachment of the cellulosome to its substrate. A scaffoldin or cellulose integrating protein may comprise one or both of such domains.

A catalase; the term “catalase” means a hydrogen-peroxide: hydrogen-peroxide oxidoreductase (EC 1.11.1.6 or EC 1.11.1.21) that catalyzes the conversion of two hydrogen peroxides to oxygen and two waters. Catalase activity can be determined by monitoring the degradation of hydrogen peroxide at 240 nm based on the following reaction: 2H₂O₂→2H₂O+O₂. The reaction is conducted in 50 mM phosphate pH 7.0 at 25° C. with 10.3 mM substrate (H₂O₂) and approximately 100 units of enzyme per ml. Absorbance is monitored spectrophotometrically within 16-24 seconds, which should correspond to an absorbance reduction from 0.45 to 0.4. One catalase activity unit can be expressed as one micromole of H₂O₂ degraded per minute at pH 7.0 and 25° C.

The term “amylase” as used herein means enzymes that hydrolyze alpha-1,4-glucosidic linkages in starch, both in amylose and amylopectin, such as alpha-amylase (EC 3.2.1.1), beta-amylase (EC 3.2.1.2), glucan 1,4-alpha-glucosidase (EC 3.2.1.3), glucan 1,4-alpha-maltotetraohydrolase (EC 3.2.1.60), glucan 1,4-alpha-maltohexaosidase (EC 3.2.1.98), glucan 1,4-alpha-maltotriohydrolase (EC 3.2.1.116) and glucan 1,4-alpha-maltohydrolase (EC 3.2.1.133), and enzymes that hydrolyze alpha-1,6-glucosidic linkages, being the branch-points in amylopectin, such as pullulanase (EC 3.2.1.41) and limit dextinase (EC 3.2.1.142).

An enzyme composition may be composed of a member of each of the classes of enzymes mentioned above, several members of one enzyme class, or any combination of these enzyme classes. Different enzymes in an enzyme composition as described herein may be obtained from different sources.

In the uses and processes described herein, the components of the compositions described above may be provided concomitantly (i.e. as a single composition per se) or separately or sequentially.

In an embodiment the enzymes in the enzyme composition are derived from a fungus, preferably a filamentous fungus or the enzymes comprise a fungal enzyme, preferably a filamentous fungal enzyme. In an embodiment a core set of (ligno)cellulose degrading enzymes (i.e. cellulases and/or a hemicellulases and/or a pectinases) may be derived from Rasamsonia emersonii. If needed, the set of enzymes can be supplemented with additional enzyme activities from other sources. Such additional activities may be derived from classical sources and/or produced by genetically modified organisms. Thus, the enzyme composition may comprise a cellulase and/or a hemicellulase and/or a pectinase from a source other than Rasamsonia. In an embodiment they may be used together with one or more Rasamsonia enzymes or they may be used without additional Rasamsonia enzymes being present.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes, Trichoderma and Trichophyton.

Several strains of filamentous fungi are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL). Examples of such strains include Aspergillus niger CBS 513.88, Aspergillus oryzae ATCC 20423, IFO 4177, ATCC 1011, ATCC 9576, ATCC14488-14491, ATCC 11601, ATCC12892, Penicillium chrysogenum CBS 455.95, Penicillium citrinum ATCC 38065, Penicillium chrysogenum P2, Talaromyces emersonii CBS 393.64, Acremonium chrysogenum ATCC 36225 or ATCC 48272, Trichoderma reesei ATCC 26921 or ATCC 56765 or ATCC 26921, Aspergillus sojae ATCC11906, Chrysosporium lucknowense C1, Garg 27K, VKM F-3500-D, ATCC44006 and derivatives thereof.

The enzymes (for example in the form of a whole fermentation broth) may be prepared by fermentation of a suitable substrate with a suitable microorganism, e.g. a filamentous fungus, wherein the enzymes are produced by the microorganism. The microorganism may be altered to improve or to make the enzymes. For example, the microorganism may be mutated by classical strain improvement procedures or by recombinant DNA techniques. Therefore, the microorganisms mentioned herein can be used as such to produce the enzymes or may be altered to increase the production or to produce altered enzymes, which might include heterologous enzymes, e.g. cellulases and/or hemicellulases and/or pectinases, thus enzymes that are not originally produced by that microorganism. Preferably, a fungus, more preferably a filamentous fungus, is used to produce the enzymes. Advantageously, a thermophilic or thermotolerant microorganism is used. Optionally, a substrate is used that induces the expression of the enzymes by the enzyme producing microorganism.

In an embodiment the enzyme composition is a whole fermentation broth. In an embodiment the enzyme composition is a whole fermentation broth of a fungus, preferably a filamentous fungus, preferably of the genus Rasamsonia. The whole fermentation broth can be prepared from fermentation of non-recombinant and/or recombinant filamentous fungi. In an embodiment the filamentous fungus is a recombinant filamentous fungus comprising one or more genes which can be homologous or heterologous to the filamentous fungus. In an embodiment, the filamentous fungus is a recombinant filamentous fungus comprising one or more genes which can be homologous or heterologous to the filamentous fungus, wherein the one or more genes encode enzymes that can degrade a cellulosic substrate. The whole fermentation broth may comprise any of the polypeptides described herein or any combination thereof.

Preferably, the enzyme composition is a whole fermentation broth, wherein cells are killed, i.e. nonviable. In an embodiment the whole fermentation broth comprises polypeptides, organic acid(s), killed cells and/or cell debris, and culture medium.

Generally, the filamentous fungi are cultivated in a cell culture medium suitable for production of enzymes capable of hydrolyzing a cellulosic substrate. The cultivation takes place in a suitable nutrient medium comprising carbon and nitrogen sources and inorganic salts, using procedures known in the art. Suitable culture media, temperature ranges and other conditions suitable for growth and cellulase and/or hemicellulase and/or pectinase production are known in the art. The whole fermentation broth can be prepared by growing the filamentous fungi to stationary phase and maintaining the filamentous fungi under limiting carbon conditions for a period of time sufficient to express the one or more cellulases and/or hemicellulases and/or pectinases. Once enzymes, such as cellulases and/or hemicellulases and/or pectinases, are secreted by the filamentous fungi into the fermentation medium, the whole fermentation broth can be used. The whole fermentation broth may comprise filamentous fungi. In an embodiment, the whole fermentation broth comprises the unfractionated contents of the fermentation materials derived at the end of the fermentation. Typically, the whole fermentation broth comprises the spent culture medium and cell debris present after the filamentous fungi are grown to saturation, incubated under carbon-limiting conditions to allow protein synthesis (particularly, expression of cellulases and/or hemicellulases and/or pectinases). In some embodiments, the whole fermentation broth comprises the spent cell culture medium, extracellular enzymes and filamentous fungi. The filamentous fungal cells present in whole fermentation broth can be killed using methods known in the art to produce a cell-killed whole fermentation broth. For instance, addition of organic acid leads to killing of the cells. If needed, the cells may also be lysed and/or permeabilized. In an embodiment, the whole fermentation broth is a cell-killed whole fermentation broth, wherein the whole fermentation broth containing the filamentous fungal cells are killed. In other words, the whole fermentation broth comprises more nonviable cells than viable cells, preferably only nonviable cells. In some embodiments, the cells are killed by lysing the filamentous fungi by chemical and/or pH treatment to generate the cell-killed whole broth of a fermentation of the filamentous fungi. In some embodiments, the cells are killed by lysing the filamentous fungi by chemical and/or pH treatment and adjusting the pH of the cell-killed fermentation mix to a suitable pH. In an embodiment, the whole fermentation broth is mixed with an organic acid.

The term “whole fermentation broth” as used herein refers to a preparation produced by cellular fermentation that undergoes no or minimal recovery and/or purification. For example, whole fermentation broths are produced when microbial cultures are grown to saturation, incubated under carbon-limiting conditions to allow protein synthesis (e.g., expression of enzymes by host cells) and secretion into cell culture medium. Typically, the whole fermentation broth is unfractionated and comprises spent cell culture medium, extracellular enzymes, and microbial, preferably nonviable, cells.

In an embodiment the whole fermentation broth can be fractionated and the one or more of the fractionated contents can be used. For instance, the killed cells and/or cell debris can be removed from a whole fermentation broth to provide an enzyme composition that is free of these components.

The whole fermentation broth may further comprise a preservative and/or anti-microbial agent. Such preservatives and/or agents are known in the art. In an embodiment the organic acid used for killing the cells can also have the function of preservative and/or anti-microbial agent.

The whole fermentation broth as described herein is typically a liquid, but may contain insoluble components, such as killed cells, cell debris, culture media components, and/or insoluble enzyme(s). In some embodiments, insoluble components may be removed to provide a clarified whole fermentation broth.

In an embodiment, the whole fermentation broth may be supplemented with one or more enzyme activities that are not expressed endogenously or expressed at relatively low level by the filamentous fungi, to improve the degradation of the cellulosic substrate, for example, to fermentable sugars such as glucose or xylose. The supplemental enzyme(s) can be added as a supplement to the whole fermentation broth, i.e. they are spiked to the whole fermentation broth. The additional enzymes may be supplemented in the form of a whole fermentation broth, or may be supplemented as purified, or minimally recovered and/or purified, enzymes.

In an embodiment, the whole fermentation broth may be supplemented with at least another whole fermentation broth. The other whole fermentation broth may be derived from the same type of fungus or from another type of fungus, e.g. a first whole fermentation broth may be derived from Rasamsonia, while a second whole fermentation broth may be derived from Rasamsonia or Aspergillus.

In an embodiment, the whole fermentation broth is a whole fermentation broth of a fermentation of a recombinant filamentous fungus overexpressing one or more enzymes to improve the degradation of the cellulosic substrate. Alternatively, the whole fermentation broth is a mixture of a whole fermentation broth of a fermentation of a non-recombinant filamentous fungus and a whole fermentation broth of a recombinant filamentous fungus overexpressing one or more enzymes to improve the degradation of the cellulosic substrate. In an embodiment, the whole fermentation broth is a whole fermentation broth of a fermentation of a filamentous fungus overexpressing beta-glucosidase. Alternatively, the whole fermentation broth is a mixture of a whole fermentation broth of a fermentation of a non-recombinant filamentous fungus and a whole fermentation broth of a fermentation of a recombinant filamentous fungus overexpressing a beta-glucosidase.

In an embodiment the enzyme composition as described herein has a pH of 2.0 to 5.5. Preferably, the enzyme composition has a pH of 2.5 to 5.0. More preferably, the enzyme composition has a pH of 3.0 to 4.5. Ergo, the enzymes in the enzyme composition are able to work at low pH.

In an embodiment the enzyme production reactor(s) used in the process for the preparation of an enzyme composition as described herein have a volume of at least 1 m³. Preferably, the enzyme production reactors have a volume of at least 1 m³, at least 2 m³, at least 3 m³, at least 4 m³, at least 5 m³, at least 6 m³, at least 7 m³, at least 8 m³, at least 9 m³, at least 10 m³, at least 15 m³, at least 20 m³, at least 25 m³, at least 30 m³, at least 35 m³, at least 40 m³, at least 45 m³, at least 50 m³, at least 60 m³, at least 70 m³, at least 75 m³, at least 80 m³, at least 90 m³. In general, the enzyme production reactor(s) will be smaller than 300 m³.

In the process for the preparation of an enzyme composition as described herein, a population of microbial cells, e.g. filamentous fungal cells, is cultured under suitable conditions for growth, in a liquid or solid medium. In an embodiment the microbial cells are cultured in a fed-batch culture, a batch culture, a continuous culture or any combination thereof. Preferably, the filamentous fungus is cultured in a fed-batch culture. A person skilled in the art is well aware of the various modes of culturing and its conditions. In an embodiment the culturing is conducted under aerobic conditions. A person skilled in the art is well aware of fermentor designs for aerobic cultivation such as for instance stirred tanks and bubble columns.

The present disclosure relates to a process for the preparation of a sugar from cellulosic material comprising the steps of (a) hydrolysing the cellulosic material with an enzyme composition to obtain the sugar, and (b) optionally, recovering the sugar, wherein the enzyme composition comprises glucoamylase and cellobiohydrolase I and the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98. All embodiments as described for the enzyme composition as described herein also apply to the process for the preparation of a sugar from cellulosic material as described herein.

The present disclosure relates to a process for the preparation of a sugar from cellulosic material comprising the steps of (a) hydrolysing the cellulosic material with an enzyme composition as described herein to obtain the sugar, and (b) optionally, recovering the sugar.

The present disclosure relates to a process for producing a fermentation product from a cellulosic material, which process comprises the steps of (a) hydrolysing the cellulosic material with an enzyme composition to obtain a sugar, (b) fermenting the obtained sugar by contacting the obtained sugar with a fermenting microorganism to produce the fermentation product, and (c) optionally, recovering the fermentation product, wherein the enzyme composition comprises glucoamylase and cellobiohydrolase I and the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98. All embodiments as described for the enzyme composition as described herein also apply to the process for producing a fermentation product from a cellulosic material as described herein.

The present disclosure also relates to a process for producing a fermentation product from a cellulosic material, which process comprises the steps of (a) hydrolysing the cellulosic material with an enzyme composition as described herein to obtain a sugar, (b) fermenting the obtained sugar by contacting the obtained sugar with a fermenting microorganism to produce the fermentation product, and (c) optionally, recovering the fermentation product.

After enzymatic hydrolysis, the hydrolysed cellulosic material may be subjected to at least one solid/liquid separation. The methods and conditions of solid/liquid separation will depend on the type of cellulosic material used and are well within the scope of the skilled artisan. Examples include, but are not limited to, centrifugation, cyclonic separation, filtration, decantation, sieving and sedimentation. In a preferred embodiment the solid/liquid separation is performed by centrifugation or sedimentation. During solid/liquid separation, means and/or aids for improving the separation may be used.

In an embodiment the cellulosic material is subjected to a pretreatment step before the enzymatic hydrolysis. In an embodiment the cellulosic material is subjected to a washing step before the enzymatic hydrolysis. In an embodiment the cellulosic material is subjected to at least one solid/liquid separation before the enzymatic hydrolysis. So, before subjecting the cellulosic material to enzymatic hydrolysis, it can be subjected to at least one solid/liquid separation. The solid/liquid separation may be done before and/or after the pretreatment step. Suitable methods and conditions for a solid/liquid separation have been described above.

In an embodiment the enzymatically hydrolysed cellulosic material is subjected to a solid/liquid separation step followed by a detoxification step and/or a concentration step.

In the processes as described herein cellulosic material may be added to the one or more hydrolysis reactors. In an embodiment the enzyme composition is already present in the one or more hydrolysis reactors before the cellulosic material is added.

In another embodiment the enzyme composition may be added to the one or more hydrolysis reactors. In an embodiment the cellulosic material is already present in the one or more hydrolysis reactors before the enzyme composition is added. In an embodiment both the cellulosic material and the enzyme composition are added simultaneously to the one or more hydrolysis reactors. The enzyme composition present in the one or more hydrolysis reactors may be an aqueous composition.

In an embodiment the enzymatic hydrolysis comprises at least a liquefaction step wherein the cellulosic material is hydrolysed in at least a first hydrolysis reactor, and a saccharification step wherein the liquefied cellulosic material is hydrolysed in the at least first hydrolysis reactor and/or in at least a second hydrolysis reactor. Saccharification can be done in the same hydrolysis reactor as the liquefaction (i.e. the at least first hydrolysis reactor), it can also be done in a separate hydrolysis reactor (i.e. the at least second hydrolysis reactor). So, in the enzymatic hydrolysis liquefaction and saccharification may be combined. Alternatively, the liquefaction and saccharification may be separate steps. Liquefaction and saccharification may be performed at different temperatures but may also be performed at a single temperature. In an embodiment the temperature of the liquefaction is higher than the temperature of the saccharification. Liquefaction is preferably carried out at a temperature of 60-85° C. and saccharification is preferably carried out at a temperature of 50-65° C.

The enzymatic hydrolysis can be performed in one or more hydrolysis reactors but can also be performed in one or more tubes or any other continuous system. This also holds true when the enzymatic hydrolysis comprises a liquefaction step and a saccharification step. The liquefaction step can be performed in one or more hydrolysis reactors but can also be performed in one or more tubes or any other continuous system and/or the saccharification step can be performed in one or more hydrolysis reactors, but can also be performed in one or more tubes or any other continuous system. Examples of hydrolysis reactors to be used include, but are not limited to, fed-batch stirred reactors, batch stirred reactors, continuous flow stirred reactors with ultrafiltration, and continuous plug-flow column reactors. Stirring can be done by one or more impellers, pumps and/or static mixers.

The enzymes used in the enzymatic hydrolysis may be added before and/or during the enzymatic hydrolysis. As indicated above, when the cellulosic material is subjected to a solid/liquid separation before enzymatic hydrolysis, the enzymes used in the enzymatic hydrolysis may be added before the solid/liquid separation. Alternatively, they may also be added after solid/liquid separation or before and after solid/liquid separation. The enzymes may also be added during the enzymatic hydrolysis. In case the enzymatic hydrolysis comprises a liquefaction step and saccharification step, additional enzymes may be added during and/or after the liquefaction step. The additional enzymes may be added before and/or during the saccharification step. Additional enzymes may also be added after the saccharification step.

In an embodiment the total enzymatic hydrolysis time is 10 hours or more, 12 hours or more, 14 hours or more, 16 hours or more, 18 hours or more, 20 hours or more, 30 hours or more, 40 hours or more, 50 hours or more, 60 hours or more, 70 hours or more, 80 hours or more, 90 hours or more, 100 hours or more, 110 hours or more, 120 hours or more, 130 hours or more, 140 hours or more, 150 hours or more, 160 hours or more, 170 hours or more, 180 hours or more, 190 hours or more, 200 hours or more.

In an embodiment, the total enzymatic hydrolysis time is 10 to 300 hours, 16 to 275 hours, preferably 20 to 250 hours, more preferably 30 to 200 hours, most preferably 40 to 150 hours.

The viscosity of the cellulosic material in the one or more hydrolysis reactors used for the enzymatic hydrolysis is between 10 and 20,000 cP, between 10 and 15,000 cP, preferably between 10 and 10,000 cP.

In case the process comprises an enzymatic hydrolysis comprising a liquefaction step and a saccharification step, the viscosity of the cellulosic material in the liquefaction step is between 10 and 4000 cP, between 10 and 2000 cP, preferably between 10 and 1000 cP and/or the viscosity of the cellulosic material in the saccharification step is preferably between 10 and 1000 cP.

The viscosity can be determined with a Rheolab QC viscosity meter using a Rushton impellor at the temperature used for the hydrolysis and at a Reynolds number <10.

In an embodiment oxygen is added during the enzymatic hydrolysis. In an embodiment oxygen is added during at least a part of the enzymatic hydrolysis. Oxygen can be added continuously or discontinuously during the enzymatic hydrolysis. In an embodiment oxygen is added one or more times during the enzymatic hydrolysis. In an embodiment oxygen may be added before the enzymatic hydrolysis, during the addition of cellulosic material to a hydrolysis reactor used for enzymatic hydrolysis, during the addition of enzyme to a hydrolysis reactor used for enzymatic hydrolysis, during a part of the enzymatic hydrolysis, during the whole enzymatic hydrolysis or any combination thereof. Oxygen is added to the one or more hydrolysis reactors used in the enzymatic hydrolysis.

Oxygen can be added in several forms. For example, oxygen can be added as oxygen gas, oxygen-enriched gas, such as oxygen-enriched air, or air. Examples how to add oxygen include, but are not limited to, addition of oxygen by means of sparging, electrolysis, chemical addition of oxygen, filling the one or more hydrolysis reactors used in the enzymatic hydrolysis from the top (plunging the hydrolysate into the tank and consequently introducing oxygen into the hydrolysate) and addition of oxygen to the headspace of said one or more hydrolysis reactors. When oxygen is added to the headspace of the hydrolysis reactor(s), sufficient oxygen necessary for the hydrolysis reaction may be supplied. In general, the amount of oxygen added to the hydrolysis reactor(s) can be controlled and/or varied. Restriction of the oxygen supplied is possible by adding only oxygen during part of the hydrolysis time in said hydrolysis reactor(s). Another option is adding oxygen at a low concentration, for example by using a mixture of air and recycled air (air leaving the hydrolysis reactor) or by “diluting” air with an inert gas. Increasing the amount of oxygen added can be achieved by addition of oxygen during longer periods of the hydrolysis time, by adding the oxygen at a higher concentration or by adding more air. Another way to control the oxygen concentration is to add an oxygen consumer and/or an oxygen generator. Oxygen can be introduced, for example blown, into the cellulosic material present in the hydrolysis reactor(s). It can also be blown into the headspace of the hydrolysis reactor.

In an embodiment oxygen is added to the one or more hydrolysis reactors used in the enzymatic hydrolysis before and/or during and/or after the addition of the cellulosic material to said one or more hydrolysis reactors. The oxygen may be introduced together with the cellulosic material that enters the hydrolysis hydrolysis reactor(s). The oxygen may be introduced into the material stream that will enter the hydrolysis reactor(s) or with part of the hydrolysis reactor(s) contents that passes an external loop of the hydrolysis reactor(s).

In an embodiment the reactor(s) used in the enzymatic hydrolysis and/or the fermentation have a volume of at least 1 m³. Preferably, the reactors have a volume of at least 1 m³, at least 2 m³, at least 3 m³, at least 4 m³, at least 5 m³, at least 6 m³, at least 7 m³, at least 8 m³, at least 9 m³, at least 10 m³, at least 15 m³, at least 20 m³, at least 25 m³, at least 30 m³, at least 35 m³, at least 40 m³, at least 45 m³, at least 50 m³, at least 60 m³, at least 70 m³, at least 75 m³, at least 80 m³, at least 90 m³, at least 100 m³, at least 200 m³, at least 300 m³, at least 400 m³, at least 500 m³, at least 600 m³, at least 700 m³, at least 800 m³, at least 900 m³, at least 1000 m³, at least 1500 m³, at least 2000 m³, at least 2500 m³. In general, the reactor(s) will be smaller than 3000 m³ or 5000 m³. In case several reactors are used in the enzymatic hydrolysis, they may have the same volume, but also may have a different volume. In case the enzymatic hydrolysis comprises a separate liquefaction step and saccharification step, the hydrolysis reactor(s) used for the liquefaction step and the hydrolysis reactor(s) used for the saccharification step may have the same volume, but also may have a different volume.

In an embodiment the enzymatic hydrolysis is done at a temperature of 40-90° C., preferably 45-80° C., more preferably 50-70° C. and most preferably 55-65° C.

Cellulosic material as used herein includes any cellulose containing material. Preferably, cellulosic material as used herein includes lignocellulosic and/or hemicellulosic material. Cellulosic material as used herein may also comprise starch and/or sucrose. Cellulosic material suitable for use in the processes as described herein includes biomass, e.g. virgin biomass and/or non-virgin biomass such as agricultural biomass, commercial organics, construction and demolition debris, municipal solid waste, waste paper and yard waste. Common forms of biomass include trees, shrubs and grasses, wheat, rye, oat, wheat straw, sugar cane, cane straw, sugar cane bagasse, switch grass, miscanthus, energy cane, cassava, molasse, barley, corn, corn stover, corn fiber, corn husks, corn cobs, canola stems, soybean stems, sweet sorghum, corn kernel including fiber from kernels, distillers dried grains (DDGS), products and by-products from milling of grains such as corn, wheat and barley (including wet milling and dry milling) often called “bran or fibre” as well as municipal solid waste, waste paper and yard waste. The biomass can also be, but is not limited to, herbaceous material, agricultural residues, forestry residues, municipal solid wastes, waste paper, and pulp and paper mill residues. “Agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, fruit peels, vines, sugar beet, sugar beet pulp, wheat midlings, oat hulls, and hard and soft woods (not including woods with deleterious materials). In addition, agricultural biomass includes organic waste materials generated from agricultural processes including farming and forestry activities, specifically including forestry wood waste. Agricultural biomass may be any of the aforementioned singularly or in any combination or mixture thereof.

In an embodiment the cellulosic material is pretreated before the enzymatic hydrolysis. Pretreatment methods are known in the art and include, but are not limited to, heat, mechanical, chemical modification, biological modification and any combination thereof. In an embodiment the pretreatment is steam treatment, dilute acid treatment, organosolv treatment, lime treatment, ARP treatment or AFEX treatment. Pretreatment is typically performed in order to enhance the accessibility of the cellulosic material to enzymatic hydrolysis and/or hydrolyse the hemicellulose and/or solubilize the hemicellulose and/or cellulose and/or lignin, in the cellulosic material. In an embodiment, the pretreatment comprises treating the cellulosic material with steam explosion, hot water treatment or treatment with dilute acid or dilute base. Examples of pretreatment methods include, but are not limited to, steam treatment (e.g. treatment at 100-260° C., at a pressure of 7-45 bar, at neutral pH, for 1-10 minutes), dilute acid treatment (e.g. treatment with 0.1-5% H₂SO₄ and/or SO₂ and/or HNO₃ and/or HCl, in presence or absence of steam, at 120-200° C., at a pressure of 2-15 bar, at acidic pH, for 2-30 minutes), organosolv treatment (e.g. treatment with 1-1.5% H₂SO₄ in presence of organic solvent and steam, at 160-200° C., at a pressure of 7-30 bar, at acidic pH, for 30-60 minutes), lime treatment (e.g. treatment with 0.1-2% NaOH/Ca(OH)₂ in the presence of water/steam at 60-160° C., at a pressure of 1-10 bar, at alkaline pH, for 60-4800 minutes), ARP treatment (e.g. treatment with 5-15% NH₃, at 150-180° C., at a pressure of 9-17 bar, at alkaline pH, for 10-90 minutes), AFEX treatment (e.g. treatment with >15% NH₃, at 60-140° C., at a pressure of 8-20 bar, at alkaline pH, for 5-30 minutes).

The cellulosic material may be washed. In an embodiment the cellulosic material may be washed after the pretreatment. The washing step may be used to remove water soluble compounds that may act as inhibitors for the fermentation and/or hydrolysis step. The washing step may be conducted in manner known to the skilled person. Next to washing, other detoxification methods do exist. The cellulosic material may also be detoxified by any (or any combination) of these methods which include, but are not limited to, solid/liquid separation, vacuum evaporation, extraction, adsorption, neutralization, overliming, addition of reducing agents, addition of detoxifying enzymes such as laccases or peroxidases, addition of microorganisms capable of detoxification of hydrolysates.

In an embodiment the hydrolysis step is conducted until 70% or more, 80% or more, 85% or more, 90% or more, 92% or more, 95% or more of available sugar in the cellulosic material is released.

In an embodiment the dry matter content of the cellulosic material in the enzymatic hydrolysis is from 10%-40% (w/w), 11%-35% (w/w), 12%-30% (w/w), 13%-29% (w/w), 14%-28% (w/w), 15%-27% (w/w), 16%-26% (w/w), 17%-25% (w/w).

In an embodiment the amount of enzyme composition added (herein also called enzyme dosage or enzyme load) in the hydrolysis is low. In an embodiment the amount of enzyme composition is 10 mg protein/g dry matter weight or lower, 9 mg protein/g dry matter weight or lower, 8 mg protein/g dry matter weight or lower, 7 mg protein/g dry matter weight or lower, 6 mg protein/g dry matter weight or lower, 5 mg protein/g dry matter or lower, 4 mg protein/g dry matter or lower, 3 mg protein/g dry matter or lower, 2 mg protein/g dry matter or lower, or 1 mg protein/g dry matter or lower (expressed as protein in mg protein/g dry matter). In an embodiment, the amount of enzyme composition is 5 mg enzyme/g dry matter weight or lower, 4 mg enzyme/g dry matter weight or lower, 3 mg enzyme/g dry matter weight or lower, 2 mg enzyme/g dry matter weight or lower, 1 mg enzyme/g dry matter weight or lower, 0.5 mg enzyme/g dry matter weight or lower, 0.4 mg enzyme composition/g dry matter weight or lower, 0.3 mg enzyme/g dry matter weight or lower, 0.25 mg enzyme/g dry matter weight or lower, 0.20 mg enzyme/g dry matter weight or lower, 0.18 mg enzyme/g dry matter weight or lower, 0.15 mg enzyme/g dry matter weight or lower or 0.10 mg enzyme/g dry matter weight or lower (expressed as total of cellulase enzymes in mg enzyme/g dry matter).

In an embodiment the enzyme composition is used in the enzymatic hydrolysis in an amount of 2 mg to 20 mg protein/gram dry matter weight of glucans in the cellulosic material. In an embodiment the enzyme composition is used in the enzymatic hydrolysis in an amount of 4.5 mg to 15 mg protein/gram dry matter weight of glucans in the cellulosic material. In an embodiment the enzyme composition is used in the enzymatic hydrolysis in an amount of 5 mg to 12 mg protein/gram dry matter weight of glucans in the cellulosic material. In an embodiment the enzyme composition is used in the enzymatic hydrolysis in an amount of 6 mg to 10 mg protein/gram dry matter weight of glucans in the cellulosic material.

As described above, the present disclosure also relates to a process for producing a fermentation product from a cellulosic material, which process comprises the steps of (a) hydrolysing the cellulosic material with an enzyme composition to obtain a sugar, (b) fermenting the obtained sugar by contacting the obtained sugar with a fermenting microorganism to produce the fermentation product, and (c) optionally, recovering the fermentation product, wherein the enzyme composition comprises glucoamylase and cellobiohydrolase I and the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98.

As described above, the present disclosure further also relates to a process for producing a fermentation product from a cellulosic material, which process comprises the steps of (a) hydrolysing the cellulosic material with an enzyme composition as described herein to obtain a sugar, (b) fermenting the obtained sugar by contacting the obtained sugar with a fermenting microorganism to produce the fermentation product, and (c) optionally, recovering the fermentation product.

In an embodiment the fermentation (i.e. step b) is performed in one or more fermentation reactors. In an embodiment the fermentation is done by an alcohol producing microorganism to produce alcohol. In an embodiment the fermentation is done by an organic acid producing microorganism to produce an organic acid. The fermentation by an alcohol producing microorganism to produce alcohol can be done in the same fermentation reactor(s) wherein the enzymatic hydrolysis is performed. Alternatively, the fermentation by an alcohol producing microorganism to produce alcohol and the fermentation by an organic acid producing microorganism to produce an organic acid can be performed in one or more separate fermentation reactors but may also be done in one or more of the same fermentation reactors.

In an embodiment the fermentation is done by a yeast. In an embodiment the alcohol producing microorganism and/or the organic acid producing microorganism is a yeast. In an embodiment the alcohol producing microorganism is able to ferment at least a C5 sugar and at least a C6 sugar. In an embodiment the organic acid producing microorganism is able to ferment at least a C6 sugar. In an embodiment the alcohol producing microorganism and the organic acid producing microorganism are different microorganisms. In another embodiment the alcohol producing microorganism and the organic acid producing microorganism are the same microorganism, i.e. the alcohol producing microorganism is also able to produce organic acid such as succinic acid.

In a further aspect, the present disclosure thus includes fermentation processes in which a microorganism is used for the fermentation of a carbon source comprising sugar(s), e.g. glucose, L-arabinose and/or xylose. The carbon source may include any carbohydrate oligo- or polymer comprising L-arabinose, xylose or glucose units, such as e.g. lignocellulose, xylans, cellulose, starch, arabinan and the like. For release of xylose or glucose units from such carbohydrates, appropriate carbohydrases (such as xylanases, glucanases, amylases and the like) may be added to the fermentation medium or may be produced by the modified host cell. In the latter case, the modified host cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases. This, in turn, will prevent repression of systems required for metabolism and transport of non-glucose sugars such as xylose. In a preferred process the modified host cell ferments both the L-arabinose (optionally xylose) and glucose, preferably simultaneously in which case preferably a modified host cell is used which is insensitive to glucose repression to prevent diauxic growth. In addition to a source of L-arabinose, optionally xylose (and glucose) as carbon source, the fermentation medium will further comprise the appropriate ingredient required for growth of the modified host cell. Compositions of fermentation media for growth of microorganisms such as yeasts or filamentous fungi are well known in the art.

The fermentation time may be shorter than in conventional fermentation at the same conditions, wherein part of the enzymatic hydrolysis still has to take part during fermentation. In one embodiment, the fermentation time is 100 hours or less, 90 hours or less, 80 hours or less, 70 hours or less, or 60 hours or less, for a sugar composition of 50 g/I glucose and corresponding other sugars from the carbohydrate material (e.g. 50 g/I xylose, 35 g/I L-arabinose and 10 g/I galactose). For more dilute sugar compositions, the fermentation time may correspondingly be reduced. In an embodiment the fermentation time of the ethanol production step is between 10 and 50 hours for ethanol made out of C6 sugars and between 20 and 100 hours for ethanol made out of C5 sugars. In an embodiment the fermentation time of the succinic acid production step is between 20 and 70 hours.

The fermentation process may be an aerobic or an anaerobic fermentation process. An anaerobic fermentation process is herein defined as a fermentation process run in the absence of oxygen or in which substantially no oxygen is consumed, preferably less than 5, 2.5 or 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem, many microorganisms use pyruvate or one of its derivatives as an electron and hydrogen acceptor thereby regenerating NAD⁺. Thus, in a preferred anaerobic fermentation process pyruvate is used as an electron (and hydrogen acceptor) and is reduced to fermentation products such as ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic and a cephalosporin. In a preferred embodiment, the fermentation process is anaerobic. An anaerobic process is advantageous, since it is cheaper than aerobic processes: less special equipment is needed. Furthermore, anaerobic processes are expected to give a higher product yield than aerobic processes. Under aerobic conditions, usually the biomass yield is higher than under anaerobic conditions. As a consequence, usually under aerobic conditions, the expected product yield is lower than under anaerobic conditions.

In another embodiment, the fermentation process is under oxygen-limited conditions. More preferably, the fermentation process is aerobic and under oxygen-limited conditions. An oxygen-limited fermentation process is a process in which the oxygen consumption is limited by the oxygen transfer from the gas to the liquid. The degree of oxygen limitation is determined by the amount and composition of the ingoing gas flow as well as the actual mixing/mass transfer properties of the fermentation equipment used. Preferably, in a process under oxygen-limited conditions, the rate of oxygen consumption is at least 5.5, more preferably at least 6 and even more preferably at least 7 mmol/L/h.

In an embodiment the alcohol fermentation process is anaerobic, while the organic acid fermentation process is aerobic, but done under oxygen-limited conditions.

The fermentation process is preferably run at a temperature that is optimal for the microorganism used. Thus, for most yeasts or fungal cells, the fermentation process is performed at a temperature which is less than 42° C., preferably 38° C. or lower. For yeast or filamentous fungal host cells, the fermentation process is preferably performed at a temperature which is lower than 35, 33, 30 or 28° C. and at a temperature which is higher than 20, 22, or 25° C. In an embodiment the alcohol fermentation step and the organic acid fermentation step are performed between 25° C. and 35° C.

In an embodiment, the fermentations are conducted with a fermenting microorganism. In an embodiment, the alcohol (e.g. ethanol) fermentations of C5 sugars are conducted with a C5 fermenting microorganism. In an embodiment, the alcohol (e.g. ethanol) fermentations of C6 sugars are conducted with a C5 fermenting microorganism or a commercial C6 fermenting microorganism. Commercially available yeast suitable for ethanol production include, but are not limited to, BIOFERM™ AFT and XR (NABC-North American Bioproducts Corporation, GA, USA), ETHANOL RED™ yeast (Fermentis/Lesaffre, USA), FALI™ (Fleischmann's Yeast, USA), FERMIOL™ (DSM Food Specialties), GERT STRAND™ (Gert Strand AB, Sweden), and SUPERSTART™ and THERMOSACC™ fresh yeast (Ethanol Technology, WI, USA).

In a preferred embodiment the fermentation product is alcohol and the fermenting microorganism is an alcohol producing microorganism that is able to ferment at least one C5 sugar.

In an embodiment propagation of the alcohol producing microorganism and/or the organic acid producing microorganism is performed in one or more propagation reactors. After propagation, the alcohol producing microorganism and/or the organic acid producing microorganism may be added to one or more fermentation reactors. Alternatively, the propagation of the alcohol producing microorganism and/or the organic acid producing microorganism is combined with the fermentation by the alcohol producing microorganism and/or the organic acid producing microorganism to produce alcohol and/or organic acid, respectively.

In an embodiment the alcohol producing microorganism is a microorganism that is able to ferment at least one C5 sugar. Preferably, it also is able to ferment at least one C6 sugar. In an embodiment the present disclosure also describes a process for the preparation of ethanol from cellulosic material, comprising the steps of (a) performing a process for the preparation of a sugar product from cellulosic material as described herein, (b) fermentation of the enzymatically hydrolysed cellulosic material to produce ethanol; and (c) optionally, recovery of the ethanol. The fermentation can be done with a microorganism that is able to ferment at least one C5 sugar.

In an embodiment the organic acid producing microorganism is a microorganism that is able to ferment at least one C6 sugar. In an embodiment the present disclosure describes a process for the preparation of succinic acid from cellulosic material, comprising the steps of (a) performing a process for the preparation of a sugar product from cellulosic material as described herein, (b) fermentation of the enzymatically hydrolysed cellulosic material to produce succinic acid; and (c) optionally, recovery of the succinic acid. The fermentation can be done with a microorganism that is able to ferment at least one C6 sugar.

The alcohol producing microorganisms may be a prokaryotic or eukaryotic organism. The microorganism used in the process may be a genetically engineered microorganism. Examples of suitable alcohol producing organisms are yeasts, for instance Saccharomyces, e.g. Saccharomyces cerevisiae, Saccharomyces pastorianus or Saccharomyces uvarum, Hansenula, Issatchenkia, e.g. Issatchenkia orientalis, Pichia, e.g. Pichia stipites or Pichia pastoris, Kluyveromyces, e.g. Kluyveromyces fagilis, Candida, e.g. Candida pseudotropicalis or Candida acidothermophilum, Pachysolen, e.g. Pachysolen tannophilus or bacteria, for instance Lactobacillus, e.g. Lactobacillus lactis, Geobacillus, Zymomonas, e.g. Zymomonas mobilis, Clostridium, e.g. Clostridium phytofermentans, Escherichia, e.g. E. coli, Klebsiella, e.g. Klebsiella oxytoca. In an embodiment the microorganism that is able to ferment at least one C5 sugar is a yeast. In an embodiment, the yeast belongs to the genus Saccharomyces, preferably of the species Saccharomyces cerevisiae. The yeast, e.g. Saccharomyces cerevisiae, used in the processes as described herein is capable of converting hexose (C6) sugars and pentose (C5) sugars. The yeast, e.g. Saccharomyces cerevisiae, used in the processes as described herein can anaerobically ferment at least one C6 sugar and at least one C5 sugar. For example, the yeast is capable of using L-arabinose and xylose in addition to glucose anaerobically. In an embodiment, the yeast is capable of converting L-arabinose into L-ribulose and/or xylulose 5-phosphate and/or into a desired fermentation product, for example into ethanol. Organisms, for example Saccharomyces cerevisiae strains, able to produce ethanol from L-arabinose may be produced by modifying a host yeast introducing the araA (L-arabinose isomerase), araB (L-ribuloglyoxalate) and araD (L-ribulose-5-P4-epimerase) genes from a suitable source. Such genes may be introduced into a host cell in order that it is capable of using arabinose. Such an approach is described in WO2003/095627. araA, araB and araD genes from Lactobacillus plantarum may be used and are disclosed in WO2008/041840. The araA gene from Bacillus subtilis and the araB and araD genes from Escherichia coli may be used and are disclosed in EP1499708. In another embodiment, araA, araB and araD genes may derived from of at least one of the genus Clavibacter, Arthrobacter and/or Gramella, in particular one of Clavibacter michiganensis, Arthrobacter aurescens, and/or Gramella forsetii, as disclosed in WO 2009011591. In an embodiment, the yeast may also comprise one or more copies of xylose isomerase gene and/or one or more copies of xylose reductase and/or xylitol dehydrogenase.

The yeast may comprise one or more genetic modifications to allow the yeast to ferment xylose. Examples of genetic modifications are introduction of one or more xy/A-gene, XYL1 gene and XYL2 gene and/or XKS1-gene; deletion of the aldose reductase (GRE3) gene; overexpression of PPP-genes TAL1, TKL1, RPE1 and RKI1 to allow the increase of the flux through the pentose phosphate pathway in the cell. Examples of genetically engineered yeast are described in EP1468093 and/or WO2006/009434.

An example of a suitable commercial yeast is RN1016 that is a xylose and glucose fermenting Saccharomyces cerevisiae strain from DSM, the Netherlands.

In an embodiment, the fermentation process for the production of ethanol is anaerobic. Anaerobic has already been defined earlier herein. In another preferred embodiment, the fermentation process for the production of ethanol is aerobic. In another preferred embodiment, the fermentation process for the production of ethanol is under oxygen-limited conditions, more preferably aerobic and under oxygen-limited conditions. Oxygen-limited conditions have already been defined earlier herein.

Alternatively, to the fermentation processes described above, at least two distinct cells may be used, this means this process is a co-fermentation process. All preferred embodiments of the fermentation processes as described above are also preferred embodiments of this co-fermentation process: identity of the fermentation product, identity of source of L-arabinose and source of xylose, conditions of fermentation (aerobic or anaerobic conditions, oxygen-limited conditions, temperature at which the process is being carried out, productivity of ethanol, yield of ethanol).

The organic acid producing microorganisms may be a prokaryotic or eukaryotic organism. The microorganism used in the process may be a genetically engineered microorganism. Examples of suitable organic acid producing organisms are yeasts, for instance Saccharomyces, e.g. Saccharomyces cerevisiae; fungi for instance Aspergillus strains, such as Aspergillus niger and Aspergillus fumigatus, Byssochlamys nivea, Lentinus degener, Paecilomyces varioti and Penicillium viniferum; and bacteria, for instance Anaerobiospirillum succiniciproducens, Actinobacillus succinogenes, Mannhei succiniciproducers MBEL 55E, Escherichia coli, Propionibacterium species, Pectinatus sp., Bacteroides sp., such as Bacteroides amylophilus, Ruminococcus flavefaciens, Prevotella ruminicola, Succcinimonas amylolytica, Succinivibrio dextrinisolvens, Wolinella succinogenes, and Cytophaga succinicans. In an embodiment the organic acid producing microorganism that is able to ferment at least one C6 sugar is a yeast. In an embodiment, the yeast belongs to the genus Saccharomyces, preferably of the species Saccharomyces cerevisiae. The yeast, e.g. Saccharomyces cerevisiae, used in the production processes of organic acid as described herein is capable of converting hexose (C6) sugars. The yeast, e.g. Saccharomyces cerevisiae, used in the processes as described herein can anaerobically ferment at least one C6 sugar.

The overall reaction time (or the reaction time of hydrolysis step and fermentation step together) may be reduced. In one embodiment, the overall reaction time is 300 hours or less, 200 hours or less, 150 hours or less, 140 hours or less, 130 or less, 120 hours or less, 110 hours or less, 100 hours of less, 90 hours or less, 80 hours or less, 75 hours or less, or about 72 hours at 90% glucose yield. Correspondingly, lower overall reaction times may be reached at lower glucose yield.

Fermentation products that may be produced by the processes as described herein can be any substance derived from fermentation. They include, but are not limited to, alcohol (such as arabinitol, butanol, ethanol, glycerol, methanol, 1,3-propanediol, sorbitol, and xylitol); organic acid (such as acetic acid, acetonic acid, adipic acid, ascorbic acid, acrylic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, maleic acid, malic acid, malonic acid, oxalic acid, oxaloacetic acid, propionic acid, succinic acid, and xylonic acid); ketones (such as acetone); amino acids (such as aspartic acid, glutamic acid, glycine, lysine, serine, tryptophan, and threonine); alkanes (such as pentane, hexane, heptane, octane, nonane, decane, undecane, and dodecane), cycloalkanes (such as cyclopentane, cyclohexane, cycloheptane, and cyclooctane), alkenes (such as pentene, hexene, heptene, and octene); and gases (such as methane, hydrogen (H₂), carbon dioxide (CO₂), and carbon monoxide (CO)). The fermentation product can also be a protein, a vitamin, a pharmaceutical, an animal feed supplement, a specialty chemical, a chemical feedstock, a plastic, a solvent, ethylene, an enzyme, such as a protease, a cellulase, an amylase, a glucanase, a lactase, a lipase, a lyase, an oxidoreductase, a transferase or a xylanase. In a preferred embodiment an organic acid and/or an alcohol is prepared in the fermentation processes as described herein. In a preferred embodiment succinic acid and/or ethanol is prepared in the fermentation processes as described herein. Preferably, the fermentation product is alcohol, preferably ethanol.

The beneficial effects as described herein are found for several cellulosic materials and therefore believed to be present for the hydrolysis of all kind of cellulosic materials. The beneficial effects are found for several enzymes and therefore believed to be present for all kind of hydrolysing enzyme compositions.

### EXAMPLES

### Example 1

### The Effect of the Ratio of GA and CBH1 on the Enzymatic Hydrolysis of Cellulosic Material

The effect of the ratio of glucoamylase (GA) and cellobiohydrolase I (CBHI) on the enzymatic hydrolysis of cellulosic material is shown in this example.

A Rasamsonia strain expressing GA was constructed and selected essentially as described in U.S. Pat. No. 9,738,890 and WO2011/054899. A composition comprising GA was produced essentially as described in WO2014/202622. The amino acid sequence of GA is shown in SEQ ID NO:2. The nucleotide sequence is shown in SEQ ID NO:1.

A composition comprising beta-glucosidase (BG) was produced essentially as described in WO2012/000890. The amino acid sequence of BG is shown in SEQ ID NO:7. The nucleotide sequence is shown in SEQ ID NO:6.

Rasamsonia emersonii enzyme cocktail was produced essentially as described in WO 2011/000949. The Rasamsonia emersonii enzyme cocktail is a whole fermentation broth comprising CBHI. The amino acid sequence of CBHI is shown in SEQ ID NO:4. The nucleotide sequence is shown in SEQ ID NO:3.

Protein concentrations of the GA composition, BG composition and enzyme cocktail were determined using a biuret method. In the biuret reaction, a copper(II) ion is reduced to copper(I), which forms a complex with the nitrogen and carbon of the peptide bonds in an alkaline solution. A violet color indicates the presence of protein. The intensity of the color, and hence the absorption at 546 nm, is directly proportional to the protein concentration, according to the Lambert-Beer law. Peptides also respond in this assay. These can be largely excluded by performing a 10 kD filtration on the samples and subtracting the result of this 10 kD filtrate from the ‘as such’ samples.

Bovine serum albumin (BSA) dilutions (0.5, 1, 2, 5, 10 and 15 mg/ml) were made with water, containing 0.2 g/L Tween-80, to generate a calibration curve. The GA composition, BG composition and enzyme cocktail samples were appropriately diluted, to fall within the result of the BSA calibration line, on weight basis with water and centrifugated for 5 minutes at >14000×g. The supernatant of each diluted sample was collected and further referred to as Sample-supernatants. Next, 500 μL of the Sample-supernatants of each diluted sample was transferred to a centrifugal reaction tube containing a 10 kD filter and centrifuged as long as needed between 15-20° C. to obtain at least 200 μL of filtrate, further referred to as Sample-supernatants-10 kDflitrate.

From all Sample-supernatants, Sample-supernatants-10 kDfiltrate and BSA dilutions, 200 μL was transferred into a 1.5 mL reaction tube, to which 800 μL BioQuant Biuret reagent was added and mixed thoroughly. Next, all the mixtures were incubated at room temperature for at least 30 minutes. The absorption of the mixtures was measured at 546 nm with a water sample used as a blank measurement. Dilutions of the GA composition, BG composition and enzyme cocktail samples that gave an absorption value at 546 nm within the range of the BSA calibration line were used to calculate the total protein concentration of the samples via the BSA calibration line. The protein concentrations measured in the Sample-supernatants-10 kDfiltrate where subtracted from the protein concentrations measured in the Sample-supernatants to get to a final protein concentrations of the GA composition, BG composition and enzyme cocktail samples.

The amounts of GA and CBHI in the GA composition, BG composition and enzyme cocktail were determined as follows. Samples of the enzyme cocktail were centrifuged for 10 minutes at 20817 rcf and 4° C. and the supernatant was diluted to a final concentration of approximately 1 mg/mL protein (protein concentration was determined using the biuret method (see above)). 100 μg protein of each sample was transferred to an Eppendorf tube and a protease inhibitor mix (Protease inhibitor cocktail, HALT, Thermo Fisher Scientific) was added to the samples. Samples were subjected to disulfide bridge reduction with either DTT or TCEP and subsequent alkylation of the free cysteines with iodoacetamide. Proteins were precipitated using ice cold 20% TCA in acetone. The protein pellet was digested with trypsin followed by deglycosylation with PNGaseF, and analysed on an ultimate 3000 (column: Waters Acquity UPLC CSH C18, 130 Å, 1.7 μm, 2.1 mm×100 mm) coupled to a QExactive Plus mass spectrometer (Thermo Fisher Scientific). The obtained data were searched against an in-house constructed Rasamsonia emersonii protein database, using Proteome Discoverer 2.3. Percolator was used to filter the results to <1% False Discovery Rate. The identified proteins were quantified using the so-called top 3 method (see Silva J C et al. Mol. & Cell Proteomics 5.1: pages 144-156, 2006) and converted to % (w/w) by the molar ratio of all proteins in each sample and the molecular weight of each protein.

The enzyme cocktail contained 37% (w/w) CBHI and no GA. The GA composition contained 45% (w/w) GA and 1.1% (w/w) CBH1. The BG composition contained 2.2% (w/w) CBH1 and no GA.

An overview of the experiments and applied enzyme dosages is shown in Table 1. Based on the dosages applied for the enzyme cocktail, GA composition and BG composition and the GA and CBHI contents determined in the cocktail and compositions the R_{CBHI} and R_{GA} were calculated.

The total amount of CBHI protein dosed in mg/g dry matter (DM) divided by the sum of the total amount of CBHI protein dosed and total amount of GA protein dosed in mg/g dry mater is reflected in the following formula: R_{CBHI}=CBHI/(CBHI+GA). The total amount of GA protein dosed in mg/g dry matter divided by the sum of the total amount of GA protein dosed and total amount of CBHI protein dosed in mg/g dry mater is reflected in the following formula: R_{GA}=GA/(GA+CBHI).

The hydrolysis reactions were performed with acid-pretreated corn fiber. The pretreatment conditions of the corn fiber were: 135-137° C., 20-45 min and pH=1.8 by addition of H₂SO₄ and the resulting pretreated corn fiber was composed of ˜13% (w/w) total glucan (of which ±5-15% was present as glucose), 8.5% (w/w) total xylan (of which ±20-40% was present as xylose) and 5% (w/w) total arabinan (of which ±75-90% was present as arabinose). The pH of the material was set at pH 4.8 and each hydrolysis reaction was done with 20 g pretreated corn fiber at a final dry matter concentration of 10% (w/w), which was added to 50 mL tubes.

At the start of each experiment, 2.75 mg total protein was added per gram of dry matter and the tubes were placed in a rotating incubator at a constant temperature of 55° C. Samples were taken for analysis after 24 hours of hydrolysis. The samples were immediately centrifuged for 8 minutes at 4000×g. The supernatant was filtered over 0.20 μm nylon filters and the filtrates were stored at 4° C. until analysis for glucose content as described below.

The glucose concentrations of the samples were measured using an HPLC equipped with an Aminex HPX-87H column (Bio-Rad Laboratories) according to the NREL technical report NREL/TP-510-42623, January 2008.

The results are presented in Table 2. The results clearly show an increased glucose release in experiments wherein R_{CBHI} is 0.97, 0.94, 0.88, 0.77 and 0.66. The results also clearly show an increased glucose release in experiments wherein R_{GA} is 0.03, 0.06, 0.12, 0.23, and 0.34. From the results follows that an increased glucose release is found in experiments wherein R_{CBHI} is from 0.98 to 0.60 and R_{GA} is from 0.02 to 0.40.

TABLE 1
Overview of applied enzyme blends.

| Experiment | 1 | 2 | 3 | 4 | 5 | 6 | 7 | 8 |
|---|---|---|---|---|---|---|---|---|
| Enzyme cocktail | 2.50 | 2.44 | 2.38 | 2.25 | 2.00 | 1.75 | 1.50 | 1.25 |
| (mg total | | | | | | | | |
| protein/g DM)¹ | | | | | | | | |
| GA composition | 0.00 | 0.06 | 0.13 | 0.25 | 0.5 | 0.75 | 1.00 | 1.25 |
| (mg total | | | | | | | | |
| protein/g DM)² | | | | | | | | |
| BG composition | 0.25 | 0.25 | 0.25 | 0.25 | 0.25 | 0.25 | 0.25 | 0.25 |
| (mg total | | | | | | | | |
| protein/g DM)³ | | | | | | | | |
| Total protein | 2.75 | 2.75 | 2.75 | 2.75 | 2.75 | 2.75 | 2.75 | 2.75 |
| (mg/g DM) | | | | | | | | |
| GA (mg | 0.00 | 0.03 | 0.06 | 0.11 | 0.23 | 0.34 | 0.45 | 0.56 |
| protein/g DM) | | | | | | | | |
| CBHI (mg | 0.93 | 0.91 | 0.89 | 0.84 | 0.75 | 0.66 | 0.57 | 0.48 |
| protein/g DM) | | | | | | | | |
| R_{CBHI} = CBHI/( | 1.00 | 0.97 | 0.94 | 0.88 | 0.77 | 0.66 | 0.56 | 0.46 |
| CBHI + GA) | | | | | | | | |
| R_{GA} = GA/ | 0.00 | 0.03 | 0.06 | 0.12 | 0.23 | 0.34 | 0.44 | 0.56 |
| (GA + CBHI) | | | | | | | | |

¹The enzyme cocktail contained 37% CBHI, no GA
²The GA composition contained 45% GA and 1.1% CBHI
³The BG composition contained 2.2% CBHI, no GA

TABLE 2
Glucose release after
24 hours hydrolysis.

| Experiment | Glucose (g/L) |
|---|---|
| 1 | 12.1 |
| 2 | 12.6 |
| 3 | 12.7 |
| 4 | 13.1 |
| 5 | 13.0 |
| 6 | 12.7 |
| 7 | 12.1 |
| 8 | 11.5 |

1. An enzyme composition comprising glucoamylase (GA) and cellobiohydrolase I (CBHI), wherein the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98.

2. The enzyme composition according to claim 1, wherein the glucoamylase comprises a GH15 glucoamylase, a GH31 glucoamylase, a GH97 glucoamylase or any combination thereof.

3. The enzyme composition according to claim 1, wherein the cellobiohydrolase I comprises a GH7 cellobiohydrolase I.

4. The enzyme composition according to claim 1, wherein the enzyme composition comprises cellobiohydrolase I in an amount of 15% to 45% (w/w) of the total amount of protein in the enzyme composition.

5. The enzyme composition according to claim 1, wherein the enzyme composition comprises glucoamylase in an amount of 0.1% to 20% (w/w) of the total amount of protein in the enzyme composition.

6. The enzyme composition according to claim 1, further comprising a beta-glucosidase (BG).

7. The enzyme composition according to claim 6, wherein the enzyme composition comprises beta-glucosidase in an amount of 1% to 20% (w/w) of the total amount of protein in the enzyme composition.

8. The enzyme composition according to claim 1, which is a whole fermentation broth.

9. A process for the preparation of a sugar from cellulosic material comprising:
a) hydrolysing the cellulosic material with an enzyme composition to obtain the sugar, and
b) optionally, recovering the sugar,
wherein the enzyme composition comprises glucoamylase and cellobiohydrolase I and the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98.

10. A process for producing a fermentation product from a cellulosic material, which process comprises:
a) hydrolysing the cellulosic material with an enzyme composition to obtain a sugar,
b) fermenting the obtained sugar by contacting the obtained sugar with a fermenting microorganism to produce the fermentation product, and
c) optionally, recovering the fermentation product,
wherein the enzyme composition comprises glucoamylase and cellobiohydrolase I and the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98.

11. The process according to claim 9, wherein an enzyme composition comprising glucoamylase (GA) and cellobiohydrolase I (CBHI), wherein the glucoamylase is present at a fraction relative to the glucoamylase and the cellobiohydrolase I as defined by R_{GA}, and wherein the cellobiohydrolase I is present at a fraction relative to the cellobiohydrolase I and the glucoamylase as defined by R_{CBHI}, wherein R_{GA} is from 0.02 to 0.40 and R_{CBHI} is from 0.60 to 0.98 is used.

12. The process according to claim 9, wherein the enzyme composition is used in an amount of 2 mg to 20 mg protein/gram dry matter weight of glucans in the cellulosic material.

13. The process according to claim 9, wherein the cellulosic material is subjected to pretreatment before the enzymatic hydrolysis.

14. The process according to claim 13, wherein the pretreatment is steam treatment, dilute acid treatment, organosolv treatment, lime treatment, ARP treatment or AFEX treatment.

15. The process according to claim 10, wherein the fermentation product is alcohol and the fermenting microorganism is an alcohol producing microorganism that is able to ferment at least one C5 sugar.


SEQUENCE LISTING (7 sequences)

SEQ ID NO: 1 - DNA, 1932 bp, Rasamsonia emersonii
  Glucoamylase
atggctcctcctctgtcctatgcgctgttcgcgctcgctctcagcccggcggtcatggcagaccctctgctgaaatcgcgcgcaacggccagcctgagcagctggctggccacggagacgtcgtatgcgctcgaggctatcctgaacaacatcggctcgtcgggtgcgtgggcgcaatcagccagcccaggcatcgtcgtggccagtcccagcacgagcgatccagattactactacacctggacgcgagactcagcgctgaccttaaaggtgctgatcgacctcttcaagaacggcaactccagcctccagggcgtgatcgaggagtatatcgatgcgcaggcctatctccagacggtatccaatccgtctggcaccctgtctacgggtggcctgggggagcccaagttcaatgtcgacaaaaccgcatttaccggcagctgggggcgtccgcagcgagatggtccggccctgcgagctactgcgctgatttcttttggcgaatggctgattgataatggctattcgacgtatgcgtctgatattgtgtggcccattgtgcgcaatgatctgtcatatgtcgcacagtattggaacgagaccggttacgatctctgggaggaagtcgacgggtcttcgttctttaccgtcgccgtccagcatcgcgcgctggttgagggagccaactttgccagcgcactgggaacgtcatgcgactactgtacctctcaggctcctgagatcctctgctacctgcagtccttctggacggggtcctacatcttggccaacttccagagcagccgctcaggaaaggacgtcaacaccatcctaggcagcatccacacgttcgaccctgaagcggcctgcgatgacaccaccttccagccctgctcaccaagagcactcgcgaatcataaagtggtggtggatgccttccgagacctctacactatcaacaacaatgccgctgaaggtgtagccgtggctgtgggtcggtatcctgaagacacatactataacggcaacccgtggttcttgtgtacgcttgctgcagccgagcagctgtatgatgctctgtatcagtggaacaacattggatccattaccatcaccgacgtctctttggatttcttccaggacttgtatagcgatgccgcggtgggcacgtactcttcctcgagctccgagtacagtgcgatcgtcgatgccgtgaagacctacgccgacggattcgtgagcatcgtggaaaactatgcactgacgaatggatctctgtccgagcaatactccaagacggacgggacacaggagtccgctcgcgatctcacctggtcgtatgctgccctgctaacggccaacatgcgtcgcaactcgatagtgcccgcagcctggggtgagacggctgccagcagtgtgccggcgacctgtgtgtccacgtcggccacaggcatctacagcactgctacggacacagcctggccgagcacgttgactgcagccacaacgacagcaacagcgacgacaacgacaacgacaaccacgagcaagaccacaacaagcacaggaacatccaccatctcaacaacctcctcctcatcatcatgtacgaccccaacatccgtagccgtaaccttcaagctaaccgcaacaacctactacggagaaaacatcaagatgtcaggctcaatcccgcagctcggcgactgggacacagacgacgcagtggccctgagcgcagcaaactacacgagcactgacccgctgtggttcgtcaccatcaacctgcccgcaggcgagtcatttgagtacaagtacatccgcatcgagagcgacgggacgatcgagtgggagagtgatccgaaccggtcgtacacggttcctgcggcttgtggtgagacggctgctgttgagagcgatacgtggcggtaa

SEQ ID NO: 2 - protein, 643 aa, Rasamsonia emersonii
  Glucoamylase
MAPPLSYALFALALSPAVMADPLLKSRATASLSSWLATETSYALEAILNNIGSSGAWAQSASPGIVVASPSTSDPDYYYTWTRDSALTLKVLIDLFKNGNSSLQGVIEEYIDAQAYLQTVSNPSGTLSTGGLGEPKFNVDKTAFTGSWGRPQRDGPALRATALISFGEWLIDNGYSTYASDIVWPIVRNDLSYVAQYWNETGYDLWEEVDGSSFFTVAVQHRALVEGANFASALGTSCDYCTSQAPEILCYLQSFWTGSYILANFQSSRSGKDVNTILGSIHTFDPEAACDDTTFQPCSPRALANHKVVVDAFRDLYTINNNAAEGVAVAVGRYPEDTYYNGNPWFLCTLAAAEQLYDALYQWNNIGSITITDVSLDFFQDLYSDAAVGTYSSSSSEYSAIVDAVKTYADGFVSIVENYALTNGSLSEQYSKTDGTQESARDLTWSYAALLTANMRRNSIVPAAWGETAASSVPATCVSTSATGIYSTATDTAWPSTLTAATTTATATTTTTTTTSKTTTSTGTSTISTTSSSSSCTTPTSVAVTFKLTATTYYGENIKMSGSIPQLGDWDTDDAVALSAANYTSTDPLWFVTINLPAGESFEYKYIRIESDGTIEWESDPNRSYTVPAACGETAAVESDTWR

SEQ ID NO: 3 - DNA, 1368 bp, Rasamsonia emersonii
  Cellobiohydrolase I
atgcttcgacgggctcttcttctatcctcttctgccatccttgctgtcaaagcacagcaggctggcacggcgactgcggagaaccacccgcccctgacatggcaggagtgcaccgctcctgggagctgcacaacccagaacggtgcggtcgttcttgattcgaactggcgttgggtgcacaatgtcgggggctacaccaactgctacacgggcaacacctggaacccaacgtactgcccggacgacgtcacctgcgccgagaattgtgcgctggacggcgcggattacgagggcacctacggcgtgacttcgtccggcagcgagttgaggctcaattttgtcaccgggtcgaacgttggatctcgtctctacctgttgcaggacgacgagacctatcagatcttcaagctcctgaaccgggaattcacctttgacgtcgatgtctccaatctcccgtgcggattgaacggcgctctgtactttgttgccatggacgctgacggcggcgtgtccaagtacccgaacaacaaggctggtgccaagtacggaaccgggtattgcgattcccaatgcccacgggacctcaagtttatcgacggcgagggaaacgtcgagggctggcagccgtcttcgaacaacgccaacactgggattggcgaccacggctcgtgctgcgctgagatggatgtctgggaagccaacagcatctccaatgcggtcactccgcacccatgcgacacgccaggacagacgatgtgcgatggggatgactgcggtggcacatactctaccaaccgctatgcgggagagtgtgatcctgacggctgtgacttcaacccttaccgcatgggcaacacttctttctacgggcctggcaagattatcgacaccacccagcccttcactgttgtgacgcagttcctcactgatgacggtactgacaccggcaccctcagcgagatcaagcgcttctacatccagaacggcaaagtcattccgcagcccaactccgacatcgccggcgtgactggcaactcgatcaccaccgagttctgcactgctcagaagcaggcctttggcgacactgacgatttctccaagcacggtggactggccagcatgggagctgccatgcagcagggcatggtcctggtgatgagtttgtgggacgactatgccgcacaaatgctgtggctggattccgactacccgaccaacgcgtccgccaccacgcctggtgttgcccgaggaacatgtccgacggactcgggcgtcccatcgcaggtcgagtcgcagagccccaactcgtacgtgacgtactcgaacatcaagtttggtccgatcaactcgacctttaccgcttcataa

SEQ ID NO: 4 - protein, 455 aa, Rasamsonia emersonii
  Cellobiohydrolase I
MLRRALLLSSSAILAVKAQQAGTATAENHPPLTWQECTAPGSCTTQNGAVVLDSNWRWVHNVGGYTNCYTGNTWNPTYCPDDVTCAENCALDGADYEGTYGVTSSGSELRLNFVTGSNVGSRLYLLQDDETYQIFKLLNREFTFDVDVSNLPCGLNGALYFVAMDADGGVSKYPNNKAGAKYGTGYCDSQCPRDLKFIDGEGNVEGWQPSSNNANTGIGDHGSCCAEMDVWEANSISNAVTPHPCDTPGQTMCDGDDCGGTYSTNRYAGECDPDGCDFNPYRMGNTSFYGPGKIIDTTQPFTVVTQFLTDDGTDTGTLSEIKRFYIQNGKVIPQPNSDIAGVTGNSITTEFCTAQKQAFGDTDDFSKHGGLASMGAAMQQGMVLVMSLWDDYAAQMLWLDSDYPTNASATTPGVARGTCPTDSGVPSQVESQSPNSYVTYSNIKFGPINSTFTAS

SEQ ID NO: 5 - DNA, 1368 bp, Artificial Sequence
  Cellobiohydrolase I
atgctccgccgtgctctcctgctgagcagctctgccatcctggccgtcaaggcccagcaggctggtactgccactgctgagaaccaccctcccttgacctggcaggagtgcactgctcctggttcctgcaccactcagaacggtgctgttgtccttgacagcaactggagatgggttcacaacgtcggtggttacaccaactgctacactggcaacacctggaaccccacctactgccccgatgatgtcacctgcgctgagaactgcgctcttgacggtgccgactacgagggtacctacggtgtcacttcttctggctctgagctccgtctgaacttcgtcaccggcagcaacgtcggctctcgcctctacctcctccaggatgacgagacttaccagatcttcaagctcctcaaccgtgagttcaccttcgatgttgatgtctccaaccttccttgcggtctgaacggtgctctgtacttcgtcgccatggatgccgacggtggtgtctccaagtaccccaacaacaaggccggtgccaagtacggtaccggttactgcgacagccagtgcccccgtgacctcaagttcattgacggcgagggcaacgtcgagggctggcagccctcctccaacaacgccaacactggtatcggtgaccacggctcttgctgcgctgagatggatgtctgggaggccaactccatctccaacgccgtcaccccccacccttgcgacacccccggccagaccatgtgcgatggtgatgactgcggtggtacctactccaccaaccgctacgccggtgagtgcgaccccgatggctgcgacttcaacccctaccgcatgggcaacacctccttctacggccctggcaagatcattgacaccacccagcccttcaccgttgtcacccagttcctgaccgatgacggcaccgacactggtaccctctccgagatcaagcgcttctacatccagaacggcaaggtcatcccccagcccaactccgacatcgccggtgtcaccggcaactccatcaccactgagttctgcactgctcagaagcaggctttcggtgacaccgatgacttctccaagcacggtggtcttgcctccatgggtgctgccatgcagcagggtatggtcctggtcatgtccctctgggatgactacgctgctcagatgctctggctcgactccgactaccccaccaacgcctccgccaccactcctggtgttgctcgtggtacctgccccaccgactctggtgttcctagccaggttgagagccagtcccccaactcctacgtgacctactccaacatcaagttcggtcccatcaactccaccttcactgcatcgtaa

SEQ ID NO: 6 - DNA, 2577 bp, Rasamsonia emersonii
  Beta-glucosidase
atgaggaacgggttgctcaaggtcgccgcccttgcggccgcttcggtcgtcaatggcgagaacctggcttattcacctcccttctacccttcgccgtgggccaatggacagggcgactgggcagaggcctacgagaaggccgtcaagtttgtctcccaactcacgctggccgaaaaggtcaacctgaccaccggaactggttgggagcaggaccgatgcgtcggccaagtgggtagcatcccaagattgggcttcccaggactttgcatgcaggactctccgctgggtgttcgagacactgactacaactcggccttcccggcgggtgtcaatgtcgctgctacctggaacagggacctcgcctaccgtcgcggccaagcgatgggcgaggagcatcgcggaaaaggtgtcgacgttcagctgggccctgtggccggcccgctgggcaggtctcccgatgctggcagaaactgggaaggtttcgccccggatcccgtgctgaccggaaacatgatggcgtccaccatccagggtattcaagatgctggtgtcattgcttgcgccaagcatttcatcctctacgagcaggagcatttccgtcagggcgctcaagatggctacgatatctccgacagtatcagtgccaacgccgatgacaagactatgcacgagttgtacttgtggccttttgccgatgctgttcgcgctggcgttggttcaatcatgtgctcctacaaccaggtgaacaacagctacgcctgctccaacagctacaccatgaacaagctgctcaagagcgaattgggcttccaaggcttcgtcatgaccgactggggtggccaccacagtggtgtgggttccgctctcgctggtttggacatgtcgatgcccggagacattgccttcgacagtggcacctccttctggggcactaacctcacggttgccgtgctcaatggaagtgttcctgagtggcgtgttgatgacatggctgtccgtatcatgtccgcttattacaaggtcggccgcgaccgctacagcgtccccatcaactttgactcgtggaccctggatacctatggtcccgagcactatgcggtgggccagggcaacaccaagatcaacgagcacgttgatgttcgcggcaaccatgcagaaatcatccatgaaatcggtgctgccagcgccgtccttctcaagaacaagggtgggcttcctttgactggcaccgaacggtttgtcggtgttttcggagaggatgccggatccaacccttggggtgtgaacggctgcagtgaccgaggctgcgacaatggtacattggccatgggctggggcagtggtactgctaacttcccctacttggtgacgccggagcaggcgatcgagagagaagtcgtgtcccgaaatggaaccttcaccgccatcacggacaatggcgctcttgagcagatggcggctgtcgcctctcaggctgatgtttgcctggtcttcgccaacgccgactccggagaaggctacatcaacgtcgacggcaatgagggtgaccggaagaatctgaccctgtggcaaggggcggatcaagtcatccacaacgtcactgccaactgcaacaacaccgtcgtggtgttgcacactgtcggccccgttttgatcgatgattggtatgaccaccccaacgtcactgccattctctgggctggtcttccgggccaggagagcggtaactcgctcgtcgatgtcctctacggccgtgtcaaccctggcggaaagactccgttcacctggggacggacccgggaggattacggtgctcctctggtcctgaagccgaacaatggcaagggcgccccgcagcaggacttcactgagggtatcttcatcgactaccgtcggtttgacaagtacaacatcacccccatctacgaattcggattcggtctgagctacactacctttgagttttctgagctcaatgtgcagcctatcaatacgccgccgtacactcccgcttctggcttcaccaaggcggcgcagtcattcggcccgtcgtccaatgcttctgacaacctgtaccccagcgacattgagcgggtcccgttgtacatctacccatggctcaactccaccgatttgaaggcgtccgccaatgaccctgactatgggttgcctaacgacaaatacgttcctcccaacgccacgaacggtaacccgcagcccattaacccggctggcggtgctcctggtggcaaccctagtctctatgagcctgttgctcgggtctcagccatcatcaccaacaccggtaaggttacgggtgacgaggttcctcaactgtatgtctctcttggcggtcccgatgatgcccccaaggttcttcgtggcttcgaccgtatcacacttgcgcctggtcagcagaccttgtggacgaccaccctgacgaggcgagacatctcgaactgggaccctgtcacccagaactgggttgtgaccaactacaccaagacggtgtatgttggcaactcctcccgcaacctgcctttgcaggcaccccttaagccatatcctggaatctaa

SEQ ID NO: 7 - protein, 858 aa, Rasamsonia emersonii
  Beta-glucosidase
MRNGLLKVAALAAASVVNGENLAYSPPFYPSPWANGQGDWAEAYEKAVKFVSQLTLAEKVNLTTGTGWEQDRCVGQVGSIPRLGFPGLCMQDSPLGVRDTDYNSAFPAGVNVAATWNRDLAYRRGQAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSIMCSYNQVNNSYACSNSYTMNKLLKSELGFQGFVMTDWGGHHSGVGSALAGLDMSMPGDIAFDSGTSFWGTNLTVAVLNGSVPEWRVDDMAVRIMSAYYKVGRDRYSVPINFDSWTLDTYGPEHYAVGQGNTKINEHVDVRGNHAEIIHEIGAASAVLLKNKGGLPLTGTERFVGVFGEDAGSNPWGVNGCSDRGCDNGTLAMGWGSGTANFPYLVTPEQAIEREVVSRNGTFTAITDNGALEQMAAVASQADVCLVFANADSGEGYINVDGNEGDRKNLTLWQGADQVIHNVTANCNNTVVVLHTVGPVLIDDWYDHPNVTAILWAGLPGQESGNSLVDVLYGRVNPGGKTPFTWGRTREDYGAPLVLKPNNGKGAPQQDFTEGIFIDYRRFDKYNITPIYEFGFGLSYTTFEFSELNVQPINTPPYTPASGFTKAAQSFGPSSNASDNLYPSDIERVPLYIYPWLNSTDLKASANDPDYGLPNDKYVPPNATNGNPQPINPAGGAPGGNPSLYEPVARVSAIITNTGKVTGDEVPQLYVSLGGPDDAPKVLRGFDRITLAPGQQTLWTTTLTRRDISNWDPVTQNWVVTNYTKTVYVGNSSRNLPLQAPLKPYPGI